(19) United States
(12) Patent Application Publication
(10) Pub. No.: US 2013/0195914 A1
(43) Pub. Date: Aug. 1, 2013

(54) Expression Of Positive Sense Single Stranded RNA Virus And Uses Thereof

(71) Applicant: Arun K. Dhar, Sykesville, MD (US)

(72) Inventor: Arun K. Dhar, Sykesville, MD (US)

(73) Assignee: Viracine Therapeutics Corporation, Columbia, MD (US)

(21) Appl. No.: 13/640,625

(22) PCT Filed: Apr. 8, 2011
(86) PCT No.: PCT/US11/31779

§ 371 Date: Dec. 26, 2012

Related U.S. Application Data
(60) Provisional application No. 61/323,241, filed on Apr. 12, 2010.

(51) Int. Cl. C12N 15/866; A61K 39/29; A61K 39/12; C12N 15/64

(52) U.S. Cl. CPC C12N 15/866; C12N 15/64; A61K 39/29; A61K 39/12
USPC 424/205.1; 435/320.1; 435/91.4

(57) ABSTRACT

The invention relates to the fields of viruses, vaccines and compounds and methods for expression. In particular, the invention includes methods and agents capable of producing quantities of a vaccine to a positive sense single stranded RNA (“(+)sense RNA”)virus.

### BACKGROUND OF THE INVENTION

1. Field of the Invention

The invention relates to the fields of viruses, vaccines and compounds and methods for expression. In particular, the invention includes methods and agents capable of producing quantities of a vaccine to a positive sense single stranded RNA (“(+)sense RNA”) virus.

2. Background of the Invention

Baculoviruses represent a family of large, rod-shaped enveloped viruses with a double stranded DNA genome size of from 80-180 Kb. Baculoviruses are considered to be species-specific among invertebrates with over 600 host species described, but they are not known to infect mammalian or other vertebrate animal cells. In the 1940's they were used widely as biopesticides and since the 1990's they have been used for producing complex eukaryotic proteins in insect culture cells (e.g., Sf9) or insect larvae (e.g., lepidopteron larvae). The most widely studied baculovirus is Autographa californica multicapsid nucleopolyhedrovirus (AcMNPV), a 134 Kb genome virus with 154 open reading frames.

Use of attenuated virus is often the most effective way of vaccinating. If a commercial attenuated vaccine is to be produced by treating an infectious virus, a commercial quantity of infectious virus must be produced for treatment. Typically, infection of a host cell as a means for production of a virus kills its host cell before commercial quantities can be obtained. Accordingly, an alternative means for production of commercial quantities of an infectious or attenuated virus are needed.

### SUMMARY OF THE INVENTION

The present invention provides a baculovirus capable of infecting a facilitating host comprising a DNA sequence that codes for a functional positive sense single stranded RNA viral genome (“(+)sense RNA”) that is not capable of infecting the facilitating host, where the transcription of the DNA sequence is under the control of a single promoter.

The present invention also provides a baculovirus that infects a facilitating host comprising a DNA sequence that codes for a functional positive sense single stranded RNA virus that does not infect the facilitating host, where the expression of the DNA sequence is under the control of a single promoter. The present invention also provides a baculovirus that infects a facilitating host comprising a DNA sequence that codes for a functional (+)sense RNA virus that does not infect the facilitating host, where the expression of the DNA sequence is not under the control of a promoter derived from a host of the (+)sense RNA virus.

The present invention also includes a DNA molecule comprising a first DNA sequence coding for a baculovirus and a second DNA sequence coding for a (+)sense RNA viral genome that does not infect insect cells, where the second DNA sequence is operably linked to only one promoter. The present invention also provides a composition comprising a first virus capable of infecting a facilitating host comprising a first DNA sequence that codes for a (+)sense RNA viral genome or complement thereof that is not capable of infecting the facilitating host and a second DNA sequence coding for a baculovirus genome capable of infecting the facilitating host comprising and a third DNA sequence that codes for a structural gene of the (+)sense RNA virus, where the expression of the first DNA sequence is under the control of a single promoter.

The present invention also provides a method of amplifying a (+)sense RNA viral genome comprising: a) infecting a facilitating host cell with virus comprising a DNA molecule comprising a first DNA sequence coding for a viral genome of the facilitating host cell and a second DNA sequence coding for a (+)sense RNA viral genome that does not infect the facilitating host cell, where the second DNA sequence is operably linked to only one promoter; b) obtaining the supernatant, cell lysate, or supernatant and cell lysate of the infected facilitating host cell; and c) transducing a host cell of the (+)sense RNA virus with the supernatant, cell lysate, or supernatant and cell lysate. The present invention also provides a method of amplifying a (+)sense RNA viral genome comprising: a) infecting an insect cell with a baculovirus comprising a first DNA sequence coding for a baculovirus genome, a second DNA sequence coding for a (+)sense RNA viral genome or complement thereof; and a third DNA sequence coding for a sub-genomic component or complement thereof from the (+)sense RNA virus; where the second DNA sequence is operably linked to a single promoter; and b) obtaining (+)sense RNA viral genome from the supernatant, cell lysate, or supernatant and cell lysate of the infected insect cell.

The present invention also provides a vaccine comprising a (+)sense RNA virus containing the (+)sense RNA viral genome produced by the method of a) infecting a facilitating host cell with virus comprising a DNA molecule comprising a first DNA sequence coding for a viral genome of the facilitating host cell and a second DNA sequence coding for a (+)sense RNA viral genome that does not infect the facilitating host cell, where the second DNA sequence is operably linked to only one promoter; b) obtaining the supernatant, cell lysate, or supernatant and cell lysate of the infected facilitating host cell; and c) transducing a host cell of the (+)sense RNA virus with the supernatant, cell lysate, or supernatant and cell lysate, where the (+)sense RNA virus is inactivated before use. The present invention also provides a vaccine comprising a (+)sense RNA virus that is not capable of infecting a facilitating host comprising a viral envelope comprising proteins derived from the facilitating host. The present invention also provides a (+)sense RNA virus comprising a (+)sense RNA viral genome comprising an additional 5-200 nucleotides at the 5′ end of the (+)sense RNA viral genome relative to a naturally occurring (+)sense RNA viral genome of the same strain.

### BRIEF DESCRIPTION OF THE DRAWINGS

FIG. 1A. A schematic diagram of firefly luciferase (fLuc) reporter constructs carrying the P2, P11, and P61 promoters of IHHNV.

FIG. 1B. The expression of the firefly luciferase reporter protein compared to Renilla luciferase in Sf9 cell.

FIG. 2. A vector map of a HCV clone in a pCR®-XL-TOPO® vector.

FIG. 3. An agarose gel electrophoresis of HCV full-length plasmid DNA (HCV pCR-XL-TOPO) digested with Spe I and Xba I enzymes. ˜9.2 kb, ˜6.5 kb and ˜2.7 kb bands represent HCV genomic bands and ˜3.5 kb represents pCR®-XL-TOPO® vector band, M=1 kb ladder. Lanes 1-12 marked as Group 1 and derived from one patient and lanes 13 and 14 are marked as Group 2 and derived from a second patient.

FIG. 4. A vector map of a HCV clone in a pFastBacDual vector. HCV genome was cloned downstream of the pol promoter.

FIG. 5. A schematic diagram of pFastBacDual vector containing a full-length genome of TSV.

FIG. 6. A schematic diagram of pFastBacDual vector containing a full-length genome of TSV as well as sub-genomic fragments, protease and ORF-2.

FIG. 7. Vector map of pFastBacDual containing a full-length genome of PRRSV.

FIG. 8. A schematic representation of genome organization of West Nile virus (WNV). C=Capsid protein, PrM=pre-membrane, and seven non-structural proteins (NS1, NS2A, NS2B, NS3, NS4A, NS4B, and NS5).

FIG. 9. A schematic diagram of pFastBacDual containing a full-length genome of WNV as well as the sub-genomic fragment containing WNV protease and structural genes.

FIG. 10. A schematic diagram of pVL 1392/1393 vector containing a full-length genome of HCV under the control of polh promoter.

FIG. 11. A schematic diagram of pVL 1392/1393 containing a full-length genome of HCV as well as the sub-genomic fragments (HCV protease and structural genes).

FIG. 12. A schematic diagram of pORB vector containing a full-length genome of HCV under the control of polH promoter.

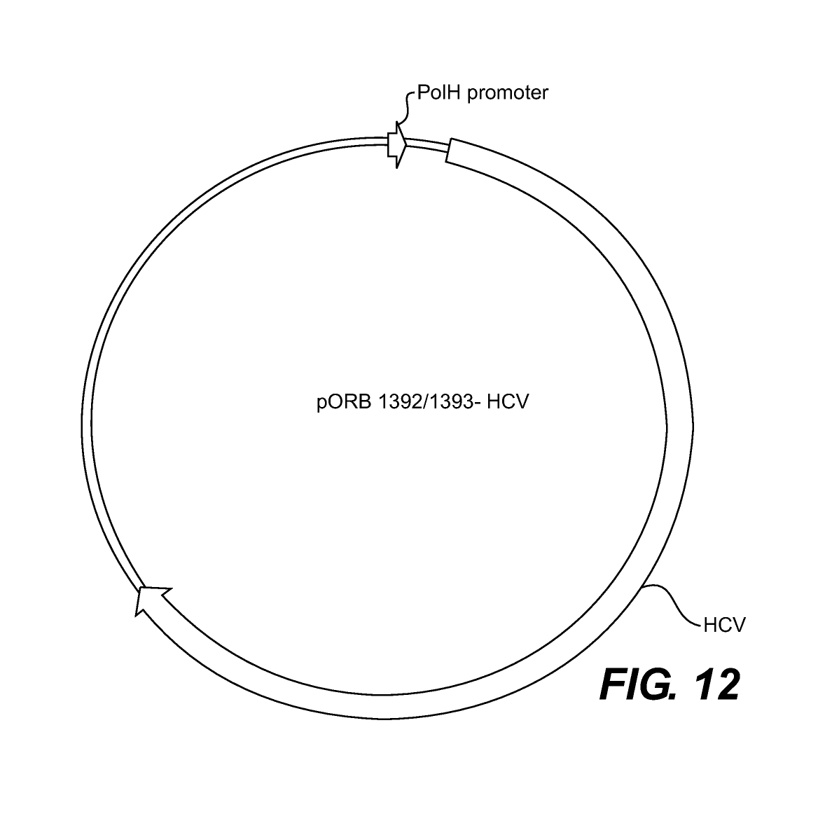

FIG. 13. A schematic diagram of pORB containing a full-length genome of HCV as well as the sub-genomic fragments (HCV protease and structural genes).

FIG. 14. A schematic diagram of pBAC™1-3 or pBAC5 or pBAC-6 vector containing a full-length genome of HCV under the control of gp64 promoter.

FIG. 15. A schematic diagram of pBAC™1-3 or pBAC5 or pBAC-6 vector containing a full-length genome of HCV as well as the sub-genomic fragments (HCV protease and structural genes).

FIG. 16. A schematic diagram of pFastBac Dual vector containing a full-length genome of HCV under the control of CMV promoter/CMV ie enhancer, or TK or P2 or ie1 promoter for the expression of HCV in mammalian cells.

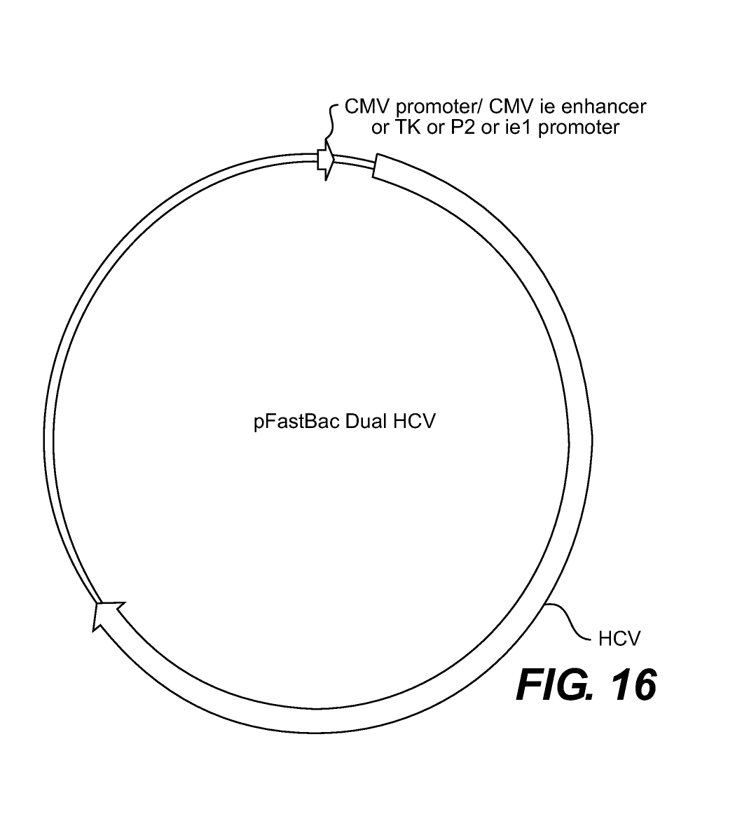

### DETAILED DESCRIPTION OF THE INVENTION

Pal et al. (J Virol 81:9339; 2007) describe the production of an insect virus (Rhopalosiphon padi; RhPV) in a homologous insect system. A second insect virus (baculovirus) was used as an expression vector for the RhPV. The RhPV was downstream from two promoters—the pot-promoter and the core baculovirus late promoter of pFastBac1.

Baculovirus can also be used to express a heterologous virus of shrimp (Taura Syndrome Virus; TSV) in an insect cell. In that case, two promoters are operably linked to the TSV viral genome. One of the promoters is the baculovirus Pol promoter and the other is a promoter homologous to the TSV host species, a promoter from the shrimp infectious hypodermal and hematopoietic necrosis virus (IHHNV) in conjunction with an Internal Ribosomal Entry Site (IRES) element (the combination is referred to as the P2 promoter). The expression of genes in heterologous systems is generally more difficult than in homologous systems, particularly if the gene sources and the heterologous system are phylogenetically distant such as insects and crustaceans. The term “heterologous” is used hereinafter for any combination of nucleic acid sequences that is not normally found intimately associated with a (+)sense RNA virus or (+)sense RNA virus host in nature. Issues of codon usage, specific and unique regulatory sequences, and post-translational modifications all need to be considered when using heterologous production systems.

One skilled in the art may refer to general reference texts for detailed descriptions of known techniques discussed herein or equivalent techniques. These texts include Ausubel et al., Current Protocols in Molecular Biology, John Wiley and Sons, Inc. (1995); Sambrook et al., Molecular Cloning, A Laboratory Manual (2d ed.), Cold Spring Harbor Press, Cold Spring Harbor, N.Y. (1989); Birren et al., Genome Analysis: A Laboratory Manual, volumes 1 through 4, Cold Spring Harbor Press, Cold Spring Harbor, N.Y. (1997-1999); R. K. Upadhyay, Advances in microbial control of insect pests, Springer; 1 edition (2003); Klein, Donald W.; Prescott, Lansing M.; Harley, John (1993). Microbiology. Dubuque, Iowa: Wm. C. Brown. ISBN 0-697-01372-3; Baculovirus and Insect Cell Expression Protocol (Methods in Molecular Biology, 2007, Second Edition, Ed. David W. Murhammer, Humana Press. These texts can, of course, also be referred to in making or using an aspect of the invention.

The present invention includes and utilizes a virus capable of infecting a facilitating host that codes for positive sense single stranded RNA viral genome (“(+)sense RNA”) RNA viral genome (“(+)sense RNA viral genome”). In an aspect of the present invention, a moncistronic, bicistronic, tricistronic or polycistronic (+)sense RNA viral genome is operably linked to only a single promoter sequence. As used herein, linked means physically linked, operably linked, or physically and operably linked. As used herein, physically linked means that the physically linked nucleic acid sequences are located on the same nucleic acid molecule, for example a facilitating viral genome can be physically linked to a moncistronic, bicistronic, tricistronic or polycistronic (+)sense viral genome as part of a single nucleic acid molecule. In a preferred aspect, only one promoter is operably linked to a DNA sequence that codes for a (+)sense RNA viral genome of the present invention such that the single promoter transcribes a single (+)sense RNA viral genome. In another aspect, a DNA sequence that codes for a moncistronic, bicistronic, tricistronic or polycistronic (+)sense RNA viral genome of the present invention is not operably linked to any promoter derived from a host of the (+)sense RNA virus.

A promoter can be any promoter. Promoters include cis-acting transcriptional control elements and regulatory sequences that are involved in regulating or modulating the timing and/or rate of transcription of a gene or genes. A promoter can be operably linked to a coding sequence for RNA polymerase to initiate transcription at the promoter and transcribe the coding sequence into RNA. In an aspect, a promoter can be pantropic, cell type-specific, tissue-specific, or a promoter functional in an insect cell, a mammalian cell, or a human cell. In a preferred aspect, the promoter is a pantropic promoter. In another aspect, a promoter is CMV, pol, p10, polH, gp64, TK, P2, P11 and P61 of IHHNV, or ie1 of white spot syndrome virus or P2, P11 and P48 of HPV. See Dhar, A. K., Kaizer, K. N., Lakshman, D. K. 2010. Transcriptional analysis of Penaeus stylirostris densovirus genes, Virology, In Press.; Dhar, A. K., Lakshman, D. K., Natarajan, S., Allnutt, F. C. T., and van Beek, N. A. M., 2007. Functional characterization of putative promoter elements from infectious hypodermal and hematopoietic necrosis virus (IHHNV) in shrimp and in insect and fish cell lines. Virus Res. 127: 1-8; Liu, W-J., Chang, Y-S., Wang, C-H., Kou, G-H., Lo, C-F. 2005. Microaray and RT-PCR screening for white spot syndrome virus immediate-early genes in cyclohexamide-treated shrimp. Virology 334: 327-341; Sukhumsirichart, W., Attasart, P., Boonsaeng, V., and Panyim, S., 2006. Complete nucleotide sequence and genome organization of hepatopancreatic parvovirus (HPV) of Penaeus monodon. Virology 346: 266-277, all hereby incorporated by reference in their entirety.

In another aspect, a promoter is derived from an insect or crustacean gene or a fish gene, a recombinant baculovirus vector or a baculovirus infecting shrimp such as Monodon baculovirus (MBV). In a further aspect, a promoter driving the transcription of a moncistronic, bicistronic, tricistronic or polycistronic (+)sense RNA viral genome is functional in a facilitating host cell, is recognized by a facilitating host RNA polymerase, or both.

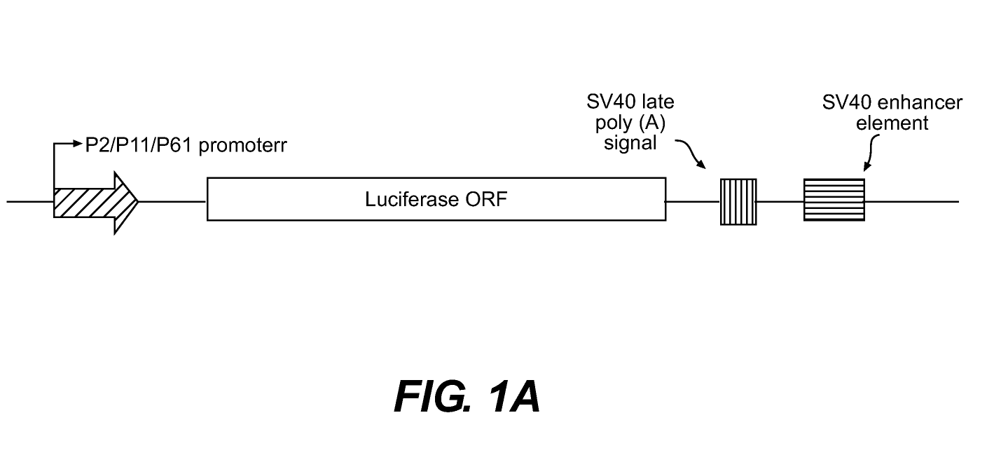

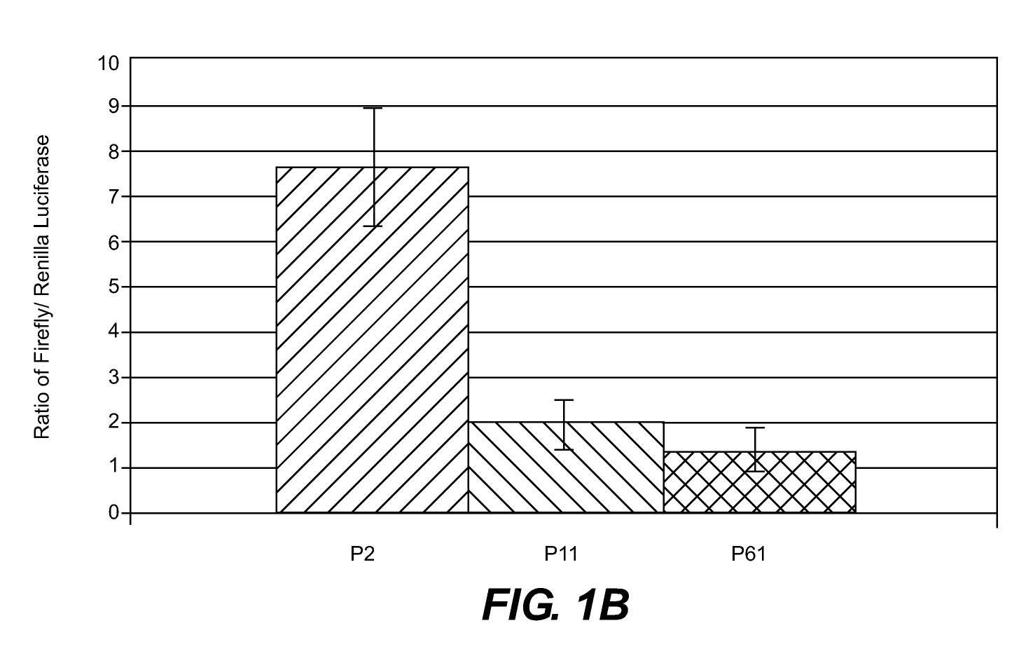

In another aspect, the promoter driving transcription of a (+)sense RNA viral genome can be selected based on strength in driving transcription. In an aspect, strength in driving transcription can be assessed by an in vitro assay before cloning the promoter upstream of the (+)sense RNA viral genome. For example, to determine the functional activities of three promoters (P2, P11, and P61) of IHHN virus of shrimp, these promoter elements can be cloned upstream of a firefly luciferase gene in a promoter assay vector, pGL3-Basic (FIG. 1A) and cotransfected with pGL4.75 hRLuc/CMV into Sf9 cells. Luciferase activities can be determined using the Dual-Glo® Luciferase Assay Kit (Promega®) following the manufacturer's recommendations. An in vitro luciferase assay can be done three times and luciferase data (fLuc/rLuc values) can be normalized to a control treatment (such as non-transfected Sf9 cells) before running paired two t-test. Normalized data can then taken to plot the bar diagram represented in FIG. 1B.

In an aspect, luciferase assays can reveal relative expression of firefly luciferase driven by different promoters. In this aspect, the relative expression of firefly luciferase is measured to a 95% confidence level of greater than p<0.05, 0.01, 0.005 or 0.001 to be significantly significance. In an aspect, a promoter driving the transcription of a (+)sense RNA viral genome has a 1.5-4 fold higher, 2-5 fold higher, 3-5 fold higher, or 4-5 fold higher level of transcription than a promoter operably linked to a subgenomic component. A promoter is selected so as when operably linked to subgenomic components, expression of said subgenomic components are optimized in order to maximize processing and virus/subcomponent(s) assembly into mature virions. For example, the P2 promoter is 3.7 fold higher than P11 and over 5-fold higher than P61 promoter. Paired two t-test shows that in vitro activity of the P2 promoter is significantly different (p<0.006) from both P11 and P61. Luciferase expression driven by the P11 promoter is 1.4 fold higher than the P61 promoter. Although there was no significant difference between these two promoters (at 95% confidence level p=0.08), there was a trend in higher luciferase expression driven by P11 promoter compared to the P61 promoter. Based on the promoter activity, P2 promoter can be operably linked to a (+)sense RNA viral genome and driving transcription.

In an aspect, a single promoter or any number of promoters operably linked to a (+)sense RNA viral genome or complement thereof is not a promoter in an infectious hypodermal and hematopoetic virus (IHHNV) of a shrimp, or has less than about 95%, 90%, 80% or 70% sequence identity to a IHHNV promoter. Percent identity can include freely available, or subscription-based algorithms including BLAST, TBLASTN, GOTOH, CLUSTAL, TBLASTX, MOTIF, or other nucleotide and/or protein sequence alignments based on the Needleman-Wunsch algorithm and/or the Smith-Waterman algorithm. In an aspect, the Needleman-Wunsch algorithm is preferred. In another aspect, a single promoter or any number of promoters operably linked to a moncistronic, bicistronic, tricistronic or polycistronic (+)sense RNA viral genome or complement thereof is heterologous to a host cell of the (+)sense RNA virus.

In an aspect, a (+)sense RNA viral genome can be the genetic material of a virus whose genetic information consists of a single strand of RNA that is the sense (or positive) strand which encodes messenger RNA (mRNA) and protein in its host cell. Replication of a functional (+)sense RNA virus in its host cell is via a negative-strand intermediate. A functional (+)sense RNA viral genome can encode a single protein which is modified by host and viral proteins to form the various proteins needed for replication and infection. One of these proteins is RNA-dependent RNA polymerase, which copies the viral RNA to form a double-stranded replicative form, which in turn directs the formation of new virions. In another aspect, a (+)sense RNA viral genome is inserted into a facilitating viral genome as a complimentary DNA copy of a (+)sense RNA viral genome. In an aspect, a (+)sense RNA viral genome is from a natural isolate or from an attenuated modification thereof. In an aspect, a (+)sense RNA viral genome can be the equivalent amount, about 75%, 75%, about 85%, 85% about 90%, 90%, about 95%, 95% about 97%, 97%, about 98%, 98%, or about 99%, 90%-95%, 80%-95%, 99%, 90%-95%, 80%-95% of an entire naturally occurring (+)sense RNA viral genome. In another aspect, a (+)sense RNA viral genome can be sufficient to produce a functional (+)sense RNA virus. In another aspect, a(+) sense RNA viral genome can be the open reading frame only. The identity can be over contiguous or noncontiguous nucleotides. In this aspect, an entire naturally occurring (+)sense RNA viral genome can be identified on GenBank on the NCBI website.

As used herein, the percent identity is preferably determined using the “Best Fit” or “Gap” program of the Sequence Analysis Software Package™ (Genetics Computer Group, Inc., University of Wisconsin Biotechnology Center, Madison, Wis.). “Gap” utilizes the algorithm of Needleman and Wunsch to find the alignment of two sequences that maximizes the number of matches and minimizes the number of gaps. Percent identity can include freely available, or subscription-based algorithms including BLAST, TBLASTN, GOTOH, CLUSTAL, TBLASTX, MOTIF, or other nucleotide and/or protein sequence alignments based on the Needleman-Wunsch algorithm and/or the Smith-Waterman algorithm. “BestFit” performs an optimal alignment of the best segment of similarity between two sequences and inserts gaps to maximize the number of matches using the local homology algorithm of Smith and Waterman. The percent identity calculations may also be performed using the Megalign program of the LASERGENE bioinformatics computing suite (default parameters, DNASTAR Inc., Madison, Wis.). The percent identity is most preferably determined using the “Best Fit” program using default parameters.

Examples of (+)sense RNA viruses include those in the order Nidovirales, such as the Family Arteriviridae, Coronaviridae (includes Coronavirus and SARS), and Roniviridae; the order Picornavirales, such as the family Dicistroviridae, Iflaviridae, Marnaviridae, Picornaviridae (includes Poliovirus, the common cold virus, Hepatitis A virus) and Secoviridae (includes subfamily Comovirinae); the order Tymovirales, such as the family Alphaflexiviridae, Betaflexiviridae, Gammaflexiviridae, Tymoviridae. Examples of (+)sense RNA virus family members further include Astroviridae, Barnaviridae, Bromoviridae, Caliciviridae (includes Norwalk virus), Closteroviridae, Flaviviridae (includes Yellow fever virus, West Nile virus, Hepatitis C virus (HCV), Dengue fever virus), Leviviridae, Luteoviridae (includes Barley yellow dwarf virus), Narnaviridae, Nodaviridae, Potyviridae, Tetraviridae, Togaviridae (includes Rubella virus, Ross River virus, Sindbis virus, Chikungunya virus), and Tombusviridae. Examples of (+)sense RNA virus family members further include the genera Benyvirus, Furovirus, Hepevirus (includes Hepatitis E virus), Hordeivirus, Idaeovirus, Ourmiavirus, Pecluvirus, Pomovirus, Sobemovirus, Tobamovirus (includes tobacco mosaic virus), Tobravirus, and Umbravirus. In an aspect, (+)sense RNA viruses include Flaviviraidae and Picornaviridae family members. Also included in an aspect of the present invention is a (+)sense RNA virus that is a Taura Syndrome Virus (TSV) or porcine reproductive and respiratory syndrome disease (PRRS or Swine flu). Further included in an aspect of the present invention is a (+)sense RNA virus that is a member of the Arbovirus family.

In an aspect, a (+)sense RNA virus can consist of a single transcript (monocistronic), two transcripts (bicistronic), three transcripts (tricistronic), or multiple transcripts (polycistronic). In this aspect, a viral genome for two or more transcripts of a (+)sense RNA virus can be operably controlled by a single or multiple promoters. As the number of transcripts increase, the choice of promoter is a more critical aspect so that a bicistronic, tricistronic or polycistronic (+)sense RNA virus having two or more transcripts will package correctly for proper infection in its host cell.

A (+)sense RNA virus, coded for by a facilitating host virus, is not capable of infecting the facilitating host or replicating independently of the facilitating virus genome inside a facilitating host cell. A functional monocistronic, bicistronic, tricistronic or polycistronic (+)sense RNA virus can infect and replicate within its native host cell. A functional (+)sense RNA virus includes structural and non-structural viral genes sufficient for infection in a monocistronic, bicistronic, tricistronic or polycistronic (+)sense RNA virus host cell. Structural and non-structural viral genes are defined in the art and are specific to a monocistronic, bicistronic, tricistronic or polycistronic (+)sense RNA virus. As used herein, the term “gene” refers to a nucleic acid sequence that can be translated to produce a polypeptide chain, or regulate gene transcription, reproduction or stability. In an aspect, genes include regions preceding and following the coding region, such as leader and trailer, promoters and enhancers, as well as, where applicable, intervening sequences (introns) between individual coding segments (exons).

In another aspect, genes additional to the (+)sense RNA viral genome or a complement thereof and the facilitating viral genome can be included in the facilitating virus. In this aspect, such additional genes can be a sub-genomic region of a monocistronic, bicistronic, tricistronic or polycistronic (+)sense RNA viral genome comprising less than the full monocistronic, bicistronic, tricistronic or polycistronic (+)sense RNA viral genome. Structural genes, non-structural genes, or a combination of structural and non-structural genes can be incorporated in the same facilitating virus operably linked to the same or a different promoter with a same or different promoter sequence than the single promoter operably linked to an entire (+)sense RNA viral genome. A structural viral gene, non-structural viral gene, or both operably linked to at least one separate promoter may improve production of a monocistronic, bicistronic, tricistronic or polycistronic (+)sense RNA virus or replication of a (+)sense RNA viral genome in a facilitating host or its own host cell. Examples of structural genes are E1 and E2 proteins in HCV or a protease gene common in many viruses. In an aspect, a facilitating viral genome includes a second, third, or greater DNA sequence that codes for a protease gene of the (+)sense RNA virus downstream from the entire monocistronic, bicistronic, tricistronic or polycistronic (+)sense RNA viral genome or another sub-genomic region.

A (+)sense RNA virus host cell can be any cell type or organism, cell culture, or larvae having a cell type that the monocistronic, bicistronic, tricistronic or polycistronic (+)sense RNA virus can infect. As used herein, infection is characterized by (+)sense RNA viral entry and viral protein expression. In an aspect, a monocistronic, bicistronic, tricistronic or polycistronic (+)sense RNA virus host cell is a mammalian cell, a human cell, a hepatoma cell, a vertebrate cell, an animal cell, a pig cell, a monkey cell, a canine cell, a mouse cell, or a eukaryotic cell. In this aspect, a monocistronic, bicistronic, tricistronic or polycistronic (+)sense RNA virus host cell can be a cell line including but not limited to Huh-7 cells, Chinese Hamster Ovary (CHO) cells, HeLa cells, Hep2G cells, primary hepatocyte cells of human or other mammalian origin, and others. In another aspect, a (+) sense RNA virus host cell can be a primary culture including but not limited to cells derived from hepatocytes. In another aspect, a monocistronic, bicistronic, tricistronic or polycistronic (+)sense RNA virus host cell is a human cell. In a further aspect, a monocistronic, bicistronic, tricistronic or polycistronic (+)sense RNA virus host cell is a human patient in need of gene therapy. In an aspect, there is more than one host cell for a (+)sense RNA virus, such as a primary and secondary host cell, where a monocistronic, bicistronic, tricistronic or polycistronic (+)sense RNA virus kills a culture of primary host cells more quickly than the same number of secondary host cells in the same culture conditions. In this aspect, a secondary host could be vaccinated with a monocistronic, bicistronic, tricistronic or polycistronic (+)sense RNA virus native in a primary host cell, such as with cowpox.

A facilitating host cell can be any cell that is not infected by the monocistronic, bicistronic, tricistronic or polycistronic (+)sense RNA virus, which genome is coded for by the facilitating virus genome. In an aspect, a facilitating host cell can be any cell type or organism, cell culture, or larvae having a cell type where the facilitating virus can replicate and infect another cell and the monocistronic, bicistronic, tricistronic or polycistronic (+)sense RNA virus cannot infect the same cell. In an aspect, a facilitating host cell can also be any whole organism-based production system, such as a crustacean or shrimp, a plant, algae, a cell from the Phyla Arthropoda, an invertebrate cell. In this aspect, a facilitating host cell is an insect cell, such as a S. frugiperda cell or Sf9 cell or any Lepidopteran insect species. In another aspect, a facilitating host cell can be a mammalian cell when a monocistronic, bicistronic, tricistronic or polycistronic (+)sense RNA virus does not infect and replicate in a mammalian cell.

In an aspect, a facilitating virus is a double-stranded DNA virus. A facilitating virus can include a virus from a family of virus such as Ascoviridae, Ascoviruses, Baculoviridae, Iridoviridae, Parvoviridae, Polydnaviridae, and Poxviridae. In an aspect, a facilitating virus is a baculovirus. In an aspect, a recombinant baculovirus can contain a monocistronic, bicistronic, tricistronic or polycistronic (+)sense RNA viral genome. The baculovirus genome can be based on a commercial vector system or other known baculovirus vectors such as, but not limited to, Sapphire™ Baculovirus, pBAC5, pBAC-6, BestBac (e.g. v-cath/chit deleted), AcNPV Baculovirus, and pFastBacDual with a monocistronic, bicistronic, tricistronic or polycistronic (+)sense RNA viral genome included. Any virus with a sufficiently large genome or packaging flexibility can be used as a facilitating virus. In an aspect, a facilitating virus is capable of transducing a monocistronic, bicistronic, tricistronic or polycistronic (+)sense RNA viral genome into a host cell of the monocistronic, bicistronic, tricistronic or polycistronic (+)sense RNA virus. As used herein, transducing refers to a facilitating virus mediating delivery of a complimentary DNA copy of a (+)sense RNA viral genome into a host cell of the monocistronic, bicistronic, tricistronic or polycistronic (+)sense RNA virus. A facilitating virus may or may not induce an immune response in the host of the monocistronic, bicistronic, tricistronic or polycistronic (+)sense RNA virus. For additional description of an adjuvant having insect cells or proteins, see U.S. Pat. No. 6,224,882 (hereby expressly incorporated in its entirety). In an aspect, the present invention includes a DNA molecule comprising a first DNA sequence coding for a baculovirus and a second DNA sequence coding for a functional monocistronic, bicistronic, tricistronic or polycistronic (+)sense RNA viral genome that does not infect insect cells, where the second DNA sequence is operably linked to only one promoter.

The term “immune response” means the reaction of the immune system when a foreign substance or microorganism enters the organism. By definition, the immune response is divided into a specific and an unspecific reaction although both are closely related. The unspecific immune response is the immediate defense against a wide variety of foreign substances and infectious agents. The specific immune response is the defense raised after a lag phase, when the organism is challenged with a substance for the first time. The specific immune response is highly efficient and is responsible for the fact that an individual who recovers from a specific infection is protected against this specific infection. Thus, a second infection with the same or a very similar infectious agent causes much milder symptoms or no symptoms at all, since there is already a “pre-existing immunity” to this agent. Such immunity and immunological memory persist for a long time, in some cases even lifelong. Accordingly, an attenuated virus of the present invention can be advantageous as a way to induce an immunological memory by vaccination. In an aspect, attenuation of a virus by treatment of a monocistronic, bicistronic, tricistronic or polycistronic (+)sense RNA virus which would otherwise have the replication and infection properties of a naturally occurring version of the same strain may be the best way to induce immunological memory.

In an aspect, a monocistronic, bicistronic, tricistronic or polycistronic (+)sense RNA virus is produced by a facilitating host cell. Following infection of a facilitating host cell with a facilitating virus, such as a baculovirus, a heterologous viral genome, such as a monocistronic, bicistronic, tricistronic or polycistronic (+)sense RNA viral genome, is transcribed in parallel with the facilitating viral genome as part of its lifecycle. In an aspect, mature virions of the facilitating virus and those of a monocistronic, bicistronic, tricistronic or polycistronic (+)sense RNA virus will be produced as translated and processed viral coat proteins combine with RNA transcribed from a complementary DNA copy of an entire monocistronic, bicistronic, tricistronic or polycistronic (+)sense RNA viral genome embedded in the facilitating viral genome, such as a recombinant baculovirus vector. In this aspect, facilitating virions, (+)sense RNA virions, or facilitating virions and (+)sense RNA virions can be obtained from the facilitating host cell. Many (+)sense RNA virions can be produced in the facilitating host regardless of how toxic the (+)sense RNA virus would be in its host cell since only the facilitating virus and not the (+)sense RNA virus will be able to infect new facilitating host cells. In an aspect, a recombinant baculovirus is able to produce 10-50 times, 50-100 times, 100-1000 times as much as 10¹⁰ times as many (+)sense RNA virions in insect cell culture as in an equivalent monocistronic, bicistronic, tricistronic or polycistronic (+)sense RNA virus host cell culture.

In an aspect, the amplification of a monocistronic, bicistronic, tricistronic or polycistronic (+)sense RNA virus genome can be measured by real-time RT-PCR. For example, Sf9 cells are infected with recombinant baculovirus containing a monocistronic, bicistronic, tricistronic or polycistronic (+)sense RNA virus genome at 100 MOI. Total RNA can be isolated at 0, 24, 48, 72, 96 and 120 hours post-infection. SYBR Green Real-time RT-PCR is performed using primers designed based on the genome sequence of the (+)sense RNA virus and following published protocols. See Dhar, A. K., Bowers, R. A., Licon, K. S., Veaze, G., and Reads, B. 2009. Validation of reference genes for quantitative measurement of immune gene expression in shrimp. Molecular Immunology, 46: 1688-95; Dhar, A. K., Roux, M. and Klimpel, K. R. 2001. Detection and quantification of infectious hypodermal and haematopoietic necrosis virus (IHHNV) and white spot virus (WSV) of shrimp by real time quantitative PCR using SYBR Green chemistry. Journal of Clinical Microbiology 39: 2835-2845, hereby incorporated by reference in their entirety. Copy number of a (+)sense RNA virus at different times post-infection (at 24, 48, 72, 96 and 120 hours) can be calculated and normalized to the viral copy number at 0 hr to determine the amplification of the (+)sense RNA virus genome over time. Copy number of a monocistronic, bicistronic, tricistronic or polycistronic (+)sense RNA virus genome can also be expressed with respect to the total genomic content of a single Sf9 cell since the number of Sf9 cells seeded before infecting with the recombinant baculovirus is known.

In an aspect of the present invention, a baculovirus that infects a facilitating host has a DNA sequence that codes for a functional monocistronic, bicistronic, tricistronic or polycistronic (+)sense RNA viral genome or complement thereof that does not infect the facilitating host, where the expression of the DNA sequence is under the control of a single promoter. In a further aspect, the single promoter operably linked to the monocistronic, bicistronic, tricistronic or polycistronic (+)sense RNA viral genome or complement thereof is not derived from a host cell of the monocistronic, bicistronic, tricistronic or polycistronic (+)sense RNA virus. In another aspect, the present invention includes a composition comprising a first virus capable of infecting a facilitating host comprising a first DNA sequence that codes for a functional monocistronic, bicistronic, tricistronic or polycistronic (+)sense RNA viral genome or complement thereof that is not capable of infecting the facilitating host and a second DNA sequence coding for a baculovirus genome capable of infecting the facilitating host comprising a third DNA sequence that codes for a structural gene of the monocistronic, bicistronic, tricistronic or polycistronic (+)sense RNA virus, where the expression of the first DNA sequence is under the control of a single promoter. Optionally in such an aspect, a second DNA sequence further comprises a fourth DNA sequence that codes for a non-structural gene of the (+)sense RNA virus, where the fourth DNA sequence is under the control of one or more promoters.

In an aspect of the present invention, a functional monocistronic, bicistronic, tricistronic or polycistronic (+)sense RNA virus can be attenuated or killed for use as a vaccine. In an aspect of the present invention, a vaccine may cause an antibody-mediated immune response, cell-mediated immunity, or both in the monocistronic, bicistronic, tricistronic or polycistronic (+)sense RNA virus host. The present invention includes a vaccine containing a baculovirus capable of infecting a facilitating host comprising a DNA sequence that codes for a monocistronic, bicistronic, tricistronic or polycistronic (+)sense RNA virus that is not capable of infecting the facilitating host, where the expression of monocistronic, bicistronic, tricistronic or polycistronic (+)sense RNA viral genome is under the control of a single promoter. In another aspect, a vaccine included in the present invention has a monocistronic, bicistronic, tricistronic or polycistronic (+)sense RNA virus, which can be in an attenuated form, that is not capable of infecting a facilitating host and has a viral envelope comprising proteins from the facilitating host. The origin of the envelope proteins can be determined based on comparison of glycosylation patterns and fatty acid profiles of the different host cells. The present invention also includes a vaccine containing a DNA molecule that codes for a baculovirus capable of infecting a facilitating host and codes for a monocistronic, bicistronic, tricistronic or polycistronic (+)sense RNA viral genome or complement thereof that is not capable of infecting the facilitating host, where the expression of (+)sense RNA viral genome is under the control of a single promoter. In an aspect, a DNA vaccine of the present invention has a DNA molecule comprising a first DNA sequence that codes for a facilitating host cell virus comprising structural and non-structural genes sufficient for infection in a facilitating host cell and a second DNA sequence that codes for a monocistronic, bicistronic, tricistronic or polycistronic (+)sense RNA viral genome or complement thereof that comprises structural and non-structural genes sufficient for infection of a host cell of a (+)sense RNA virus, but not sufficient for infection of a facilitating host cell, where the expression of the monocistronic, bicistronic, tricistronic or polycistronic (+)sense RNA viral genome or complement thereof is under the control of a single promoter.

In another aspect of the present invention, a monocistronic, bicistronic, tricistronic or polycistronic (+)sense RNA virus can be used directly or after purification from the facilitating host cell, host cell proteins, or both. A (+)sense RNA virus that has an envelope coat can have envelope proteins from the facilitating host cell, rather than the monocistronic, bicistronic, tricistronic or polycistronic (+)sense RNA virus host cell or a native host cell. In an aspect, a vaccine containing such a virus would have the envelope coat proteins from a facilitating host cell, such as an insect cell. This could be an advantage due to an increase in stimulating an immune response in a host cell. See U.S. Pat. No. 6,224,882, hereby explicitly incorporated in its entirety by reference. In an aspect a vaccine may include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

For the preparation of vaccines, an attenuated or killed monocistronic, bicistronic, tricistronic or polycistronic (+)sense RNA virus of the present invention is converted into a physiologically acceptable form. This can be done based on experience in the preparation of poxvirus vaccines used for vaccination against smallpox (as described by Stickl, H. et al. Dtsch. med. Wschr. 99, 2386-2392). For example, the 45 purified virus is stored at −80° C. with a titre of 5×108 TCID/ml formulated in about 10 mM Tris, 140 mM NaCl, pH 7.4. For the preparation of vaccine shots, e.g., 10²-10⁸ particles of the virus are lyophilized in 100 ml of phosphate-buffered saline (PBS) in the presence of 2% peptone and 1% 50 human albumin in an ampoule, preferably a glass ampoule. Alternatively, the vaccine shots can be produced by stepwise, freeze-drying of the virus in a formulation. This formulation can contain additional additives such as mannitol, dextran, sugar, glycine, lactose, polyvinylpyrrolidone, or other additives, such as antioxidants or inert gas, stabilizers or recombinant proteins (e.g. human serum albumin) suitable for in vivo administration. The glass ampoule is then sealed and can be stored between 4° C. and room temperature for several months. However, as long as no need exists the ampoule is stored preferably at temperatures below −20° C.

For vaccination or therapy, the lyophilisate can be dissolved in 0.1 to 0.5 ml of an aqueous solution, preferably physiological saline or Tris buffer, and administered either systemically or locally, i.e., by parenteral, intramuscular, or any other path of administration know to a skilled practitioner. The mode of administration, dose, and number of administrations can be optimized by those skilled in the art in a known manner.

In another aspect, the present invention includes a vaccine against a (+)sense RNA virus comprising a monocistronic, bicistronic, tricistronic or polycistronic (+)sense RNA viral genome or complement thereof comprising a signature nucleic acid sequence having a length of 5-200, about 100, 50-60, 25-200, 50-100 nucleotides at the 5′ end, 3′ end, or 5′ and 3′ ends of the viral genome. The signature nucleic acid sequence is an addition of nucleic acid sequence at the 5′ or 3′ end of the monocistronic, bicistronic, tricistronic or polycistronic (+)sense RNA viral genome of the present invention relative to a naturally occurring monocistronic, bicistronic, tricistronic or polycistronic (+)sense RNA viral genome of the same strain. In a particular aspect, the nucleic acid sequence of the signature is the complement of a portion of a facilitating host promoter DNA sequence. Even more specifically, the dangling bit of nucleic acid sequence is a transcription initiation site or the sequence 20-30 nucleotides downstream of the TATA box from a facilitating host promoter DNA sequence or complements thereof.

In another aspect, a (+)sense RNA virus can have envelope proteins from the (+)sense RNA virus host cell, such as a CHO cell line. Viruses that have envelopes are known in the art, such as influenza and HCV. In an aspect, a monocistronic, bicistronic, tricistronic or polycistronic (+)sense RNA virus from a facilitating host cell can infect a monocistronic, bicistronic, tricistronic or polycistronic (+)sense RNA virus host cell and the RNA viral genome will be further replicated and packaged into new (+)sense RNA virus. In an aspect, a functional or nonfunctional (+)sense RNA virus is attenuated, inactivated/killed after obtaining it from a (+)sense RNA virus host cell or facilitating host cell. Methods of attenuating a virus are known in the art as are methods of killing, such as by treatment with formalin. In an aspect, a (+)sense RNA virus is attenuated if it has reduced virulence relative to the viral genome of a naturally occurring (+)sense RNA virus of the same strain. For example, the rate of infection or replication or both with an attenuated (+)sense RNA virus is reduced compared to a naturally occurring (+)sense RNA virus of the same strain. The growth behavior or amplification/replication of a virus can be expressed by the ratio of virus produced from an infected cell (Output) to the amount originally used to infect the cell in the first place (Input) (“amplification ratio”). A ratio of “1” between Output and Input defines an amplification status where the amount of virus produced from the infected cells is the same as the amount initially used to infect the cells. Such a ratio is understood to mean that the infected cells are permissive for virus infection and virus reproduction. An amplification ratio of less than 1, i.e., a decrease of the amplification below input level, indicates a lack of reproductive replication and thus, attenuation of the virus. In a particular aspect of attenuated viruses, a monocistronic, bicistronic, tricistronic or polycistronic (+)sense RNA viral genome or complement thereof has a codon bias different than the codon bias of its native (+)sense RNA virus host cell.

In another aspect, vaccines produced using the instant invention can be delivered to vertebrates, including human, by subcutaneous injection, or via technologies know in the art for mucosal delivery of vaccines such as, but not limited to, oral or nasal delivery. In an aspect, a vaccine can be used in a vaccination program. “Vaccination” means that a monocistronic, bicistronic, tricistronic or polycistronic (+)sense RNA virus host is challenged with (+)sense RNA virus of the present invention, e.g., an attenuated or inactivated form of a (+)sense RNA virus, to induce a specific immunity. A specific immune response against a monocistronic, bicistronic, tricistronic or polycistronic (+)sense RNA virus may be induced. The (+)sense RNA virus host, thus, is immunized, or has immunity, against the (+)sense RNA virus.

“Immunity” means partial or complete protection of a monocistronic, bicistronic, tricistronic or polycistronic (+)sense RNA virus host against diseases caused by the (+)sense RNA virus due to a successful elimination of a preceding infection with the monocistronic, bicistronic, tricistronic or polycistronic (+)sense RNA virus or a characteristic part thereof. Immunity is based on the existence, induction, and activation of specialized cells of the immune system.

In an aspect, small or large scale fermentor operations known in the art can be used to produce 10⁴-10¹⁰, 10⁵-10⁹, 10⁶-10⁹, 10⁶-10¹⁰, 10⁷-10⁹ (+)sense RNA virions. In a further aspect, operations can include growth of a facilitating host cell in a range of temperature such as 20° C. to 35° C., 23° C. to 30° C., 24° C. to 29° C., or 20° C. to 30° C. In this aspect, growth of a (+)sense RNA host cell is not optimal. In another aspect, a temperature-sensitive mutant of a facilitating host cell, such as Sf9 cells, may be used to enhance and/or optimize cell growth. In another aspect, cell harvest virus purification is around 5 days, 5 days, about 3-8 days, 1-7 days, 2-6 days, or 5-7 days. Reusable fermentation devices from roller bottles to stirred tank fermentors can be used. Alternatively, single use fermentation systems such as, but not limited to, the WAVE bioreactor (Invitrogen Inc; Carlsbad, Calif.) or Flex-Factory biomanufacturing platform (Xcellerex Inc. Marlborough, Mass.) can also be used. Advantages of the later involve portability, such that the viral vaccine can be manufactured at remote locations that may be more amenable to the delivery of vaccines to the patients. Alternatively, the baculovirus can be used to directly infect lepidopteron larvae for the production of the RNA virus in a whole organism. In this latter case, the lepidopteron larvae would be grown in facilities designed for such a purpose of producing baculovirus-based recombinant vaccines (e.g., Chesapeake PERL; 8510A Corridor Road, Savage, Md. 20763).

In an aspect of a method included in the present invention further includes a method of amplifying a monocistronic, bicistronic, tricistronic or polycistronic (+)sense RNA viral genome comprising: a) infecting a facilitating host cell with virus comprising a DNA molecule comprising a first DNA sequence coding for a viral genome of the facilitating host cell and a second DNA sequence coding for a positive strand ssRNA viral genome that does not infect the facilitating host cell, where the second DNA sequence is operably linked to only one promoter; b) obtaining the supernatant, cell lysate, or supernatant and cell lysate of the infected facilitating host cell; and c) transducing a host cell of the (+)sense RNA virus with the supernatant, cell lysate, or supernatant and cell lysate. In this aspect when making a baculovirus clone, the infection is at an MOI between 0.1-10 for 24 to 72 hours, preferably 72 hours on a monolayer of facilitating host cells. In a separate aspect, the infection for commercial production of (+)sense RNA virus is at an MOI of 1-10,000, 10-100, 1-100, 50-100 for about 72 hours in a suspension culture of 10⁶-10⁹ the facilitating host cells. Another aspect of a method included in the present invention includes transducing a monocistronic, bicistronic, tricistronic or polycistronic (+)sense RNA viral host cell with a baculovirus having a (+)sense RNA viral genome under the control of a single promoter. The transduction by baculovirus has better delivery of (+)sense RNA than transfection of (+)sense RNA viral genome or complement thereof without the baculovirus component.

In a preferred aspect, the facilitating host cells are insect cells. In a further aspect of a method of the present invention, the supernatant, cell lysate, or supernatant and cell lysate of the host cell of the monocistronic, bicistronic, tricistronic or polycistronic (+)sense RNA virus is obtained and is capable of producing antibodies in mice that cross react with a naturally occurring form of the (+)sense RNA virus. In a further aspect of a method of the present invention, the supernatant, cell lysate, or supernatant and cell lysate of the facilitating host cells can be obtained and is capable of producing antibodies in mice that cross react with a naturally occurring form of the positive strand ssRNA virus. In another aspect of a method of the present invention, the supernatant, cell lysate, or supernatant and cell lysate of the host cell of the (+)sense RNA virus is obtained and any one or more of these is capable of replicating and propagating in a mammalian cell line.

In a different aspect, the present invention includes a method of making a baculovirus vector containing a DNA sequence of a monocistronic, bicistronic, tricistronic or polycistronic (+)sense RNA viral genome or complement thereof by obtaining a DNA sequence of a (+)sense RNA virus; and cloning the DNA sequence into a baculovirus vector, where the DNA sequence of a (+)sense RNA virus or complement thereof is operably linked to a single promoter. Another aspect of the present invention includes a method of amplifying a monocistronic, bicistronic, tricistronic or polycistronic (+)sense RNA viral genome by transfecting an insect cell with a DNA molecule comprising a first DNA sequence coding for an insect viral genome and a second DNA sequence coding for a monocistronic, bicistronic, tricistronic or polycistronic (+)sense RNA viral genome or complement thereof; and a third DNA sequence that codes a sub-genomic component of the (+)sense RNA virus; where the second DNA sequence coding for a (+)sense RNA viral genome or complement thereof is operably linked to a single promoter; obtaining supernatant, cell lysate, or supernatant and cell lysate of progeny of the transfected insect cell; and isolation of the (+)sense RNA virus.

In a further aspect, a method of the present invention includes producing a monocistronic, bicistronic, tricistronic or polycistronic (+)sense RNA virus by expressing in a facilitating host cell a DNA molecule with a first DNA sequence coding for a portion of a virus of the facilitating host cell and a second DNA sequence coding for a (+)sense RNA viral genome or complement thereof that does not infect the facilitating host cell, where the second DNA sequence is operably linked to single promoter; obtaining a monocistronic, bicistronic, tricistronic or polycistronic (+)sense RNA virus that does not infect the facilitating host cell. Optionally, this method can include a third DNA sequence on the DNA molecule that codes for a sub-genomic component of the (+)sense RNA virus, under the control of a second promoter that has the same or different sequence from the promoter operably linked to the second DNA sequence.

In a further aspect, a method of the present invention includes producing a vaccine by expressing in a facilitating host cell a baculovirus comprising a DNA sequence that encodes a monocistronic, bicistronic, tricistronic or polycistronic (+)sense viral genome or complement thereof; and amplification of the monocistronic, bicistronic, tricistronic or polycistronic (+)sense viral genome or complement thereof inside the facilitating host cell. The monocistronic, bicistronic, tricistronic or polycistronic (+)sense viral genome or complement thereof can serve as the vaccine or, in another aspect, the facilitating host cell can package the (+)sense viral genome or complement thereof for production of a (+)sense RNA virus and that virus can be inactivated or attenuated.

In an aspect, the present invention includes a pFastBac® Dual vector with a monocistronic, bicistronic, tricistronic or polycistronic (+)sense RNA viral genome, such as HCV, or a complement thereof downstream of only the pol promoter. In another aspect, the present invention includes a pFastBac® Dual vector with a monocistronic, bicistronic, tricistronic or polycistronic (+)sense RNA viral genome, such as HCV, or a complement thereof downstream of only the pol promoter and a sub-genomic component, such as the structural genes of HCV, operably linked to the p10 promoter. In a further aspect, the present invention includes a pFastBac® Dual vector with a (+)sense RNA viral genome, such as a monocistronic (+)sense RNA virus like HCV, or a complement thereof downstream of the pol promoter only and a sub-genomic component, such as the structural genes of HCV, operably linked to a p10 promoter; and a protease gene, such as the HCV protease gene, under the control of a p2 promoter downstream of the sub-genomic component of structural genes. In another aspect, the present invention includes a pFastBac® Dual vector with a (+)sense RNA viral genome, such as a monocistronic (+)sense RNA virus like TSV, or a complement thereof operably linked to the pol promoter; downstream of that is a p2 promoter operably linked to TSV structural genes, such as ORF-2; further downstream is a p10 promoter operably linked to a TSV protease. In a further aspect of the present invention, a pFastBac® Dual vector with a (+)sense RNA viral genome, such as TSV, or a complement thereof operably linked to the pol promote is upstream of a sub-genomic component comprising the ORF-2, or optionally ORF-1, through ORF-7 of the PRRSV genome under the control of the p10 promoter.

In a different aspect, the present invention includes a pFastBac® Dual vector with a (+)sense RNA viral genome, such as WNV, or a complement thereof operably linked to a pol promoter only; upstream of a p2 promoter operably linked to WNV structural genes, such as the first three genes; further downstream of the WNV genome is a p10 promoter operably linked to a WNV protease. In a further aspect, the present invention also includes a recombinant baculovirus vector, such as pVL1392/1393®, with a (+)sense RNA viral genome, such as HCV, or a complement thereof operably linked to a pol promoter only; upstream of a p2 promoter operably linked to WNV structural genes, such as the first three genes; further downstream of the WNV genome is a p10 promoter operably linked to a WNV protease. In yet another aspect, the present invention also includes a recombinant baculovirus vector, such as pVL1392/1393®, with a (+)sense RNA viral genome, such as HCV, or a complement thereof operably linked to a PolH promoter only; upstream of a p2 promoter operably linked to an HCV protease gene, and further downstream is a p10 promoter operably linked to HCV structural genes.

One other aspect of the present invention is a pORB vector with a full-length genome of HCV under the control of polH promoter only. A different aspect of the present invention is a pORB vector containing a full-length genome of HCV operably linked to only a polH promoter upstream of a p2 promoter operably linked to an HCV protease and further downstream from both is a p10 promoter operably linked to HCV structural genes, the position of the genes relative to each other on the vector is not essential to function. In another aspect, a vector of the present invention includes pBAC™ 1-3 or pBAC5 or pBAC-6 vector containing a full-length genome of HCV under the control of a gp64 promoter. Another vector of the present invention includes pBAC™ 1-3 or pBAC5 or pBAC-6 vector containing a full-length genome of HCV as well as the sub-genomic fragments (HCV protease and structural genes) each operably linked to a p2 or p10 promoter. Another vector of the present invention includes a pFastBac Dual® vector containing a (+)sense RNA viral genome, such as a full-length genome of HCV, under the control of CMV promoter/CMV ie enhancer, or TK or P2 or ie1 promoter.

Having now generally described the invention, the same will be more readily understood through reference to the following examples that are provided by way of illustration, and are not intended to be limiting of the present invention, unless specified.

Each periodical, patent, and other document or reference cited herein is herein incorporated by reference in its entirety.

### EXAMPLES

### Example 1

### Cloning of a Full-Length Genome of HCV in a pCR®-XL-TOPO® Vector

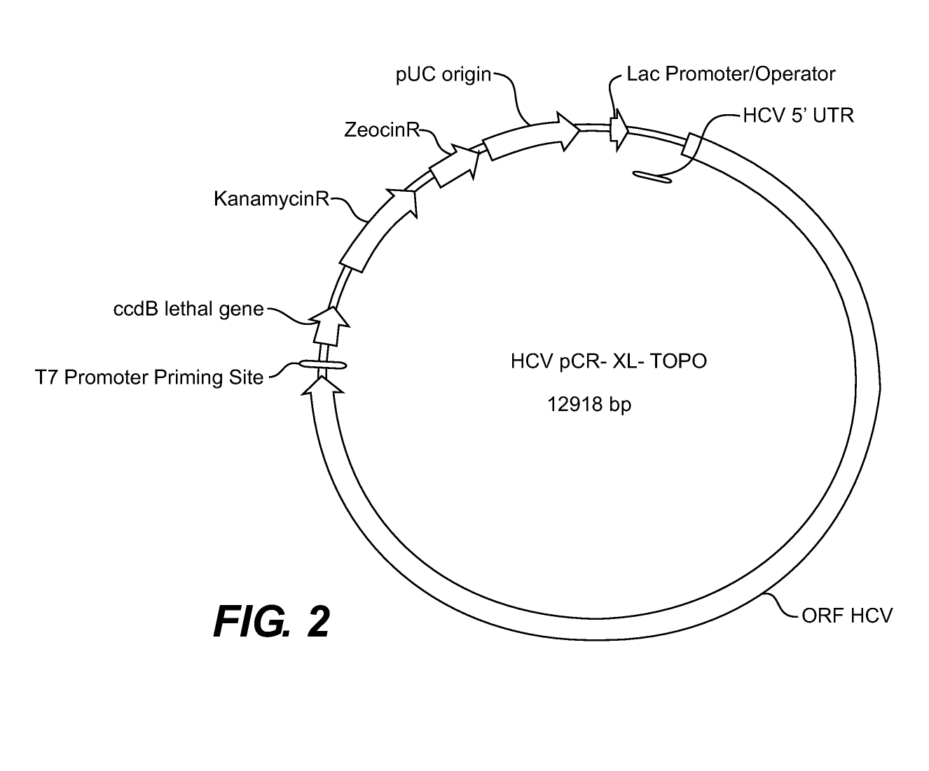

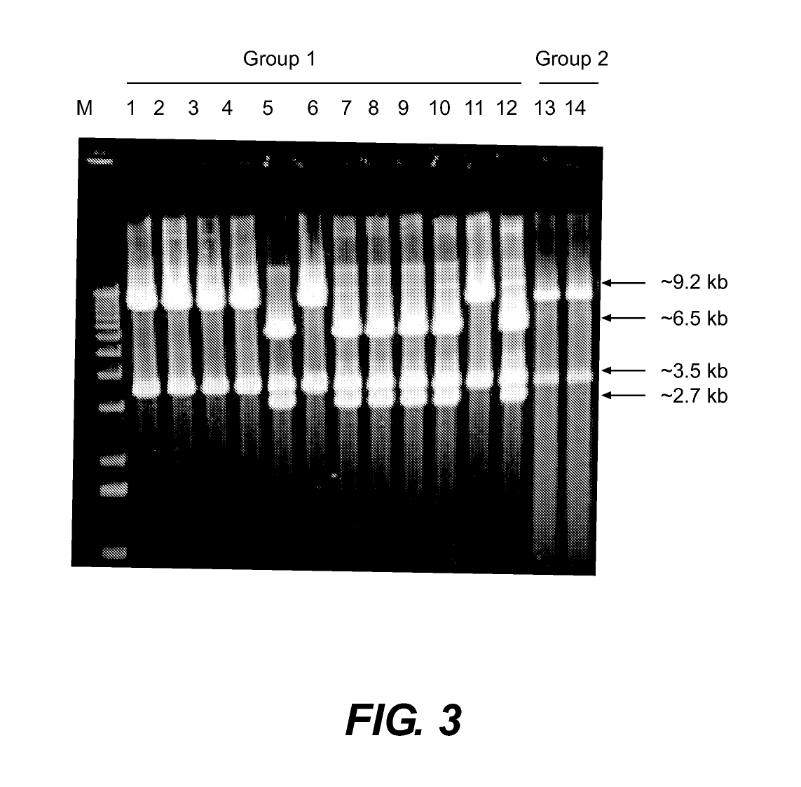

Polymerase chain reaction-amplified cDNA of a full-length HCV genome was obtained from Saint Louis University, Department of Internal Medicine, and St. Louis, Mo. 63110. The amplicon was cloned into a pCR®-XL-TOPO® vector following manufacturer's recommendations (Invitrogen Inc.®, Carlsbad, Calif.). The plasmid DNA was isolated from the recombinant clones, digested with Spe I and Xba I enzymes and run in a 1% agarose gel. A ˜9.2 kb band represents full-length HCV genome, as well as ˜6.5 kb and ˜2.7 kb band represent HCV genome with an internal restriction site. A ˜3.5 kb band represents pCR®-XL-TOPO® vector. Based on the restriction analysis, it was apparent there are at least two classes of genotypic variants in these clones. These include, lanes 1, 2, 3, 4, 6, 11, 13 and 14 represent one class, and the lanes 5, 7, 8, 9, 10 and 12 represent the second class of genomic variants. The restriction analysis data also indicated that the recombinant clones contained the full-length genome of HCV. A vector map of the HCV genome in the pCR®-XL-TOPO® vector, and a photograph of restriction analysis of HCV clones are shown in FIGS. 2 and 3, respectively.

### Example 2

### Construction of a Baculovirus Containing a Full-Length HCV Genome Under the Control of the Pol-Promoter

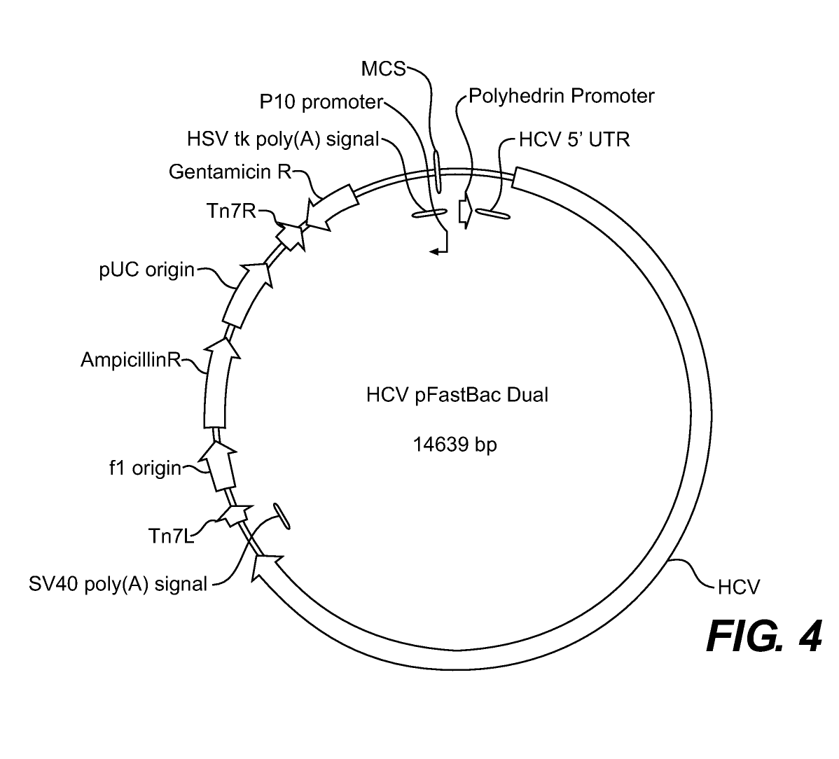

The plasmid DNA of the full-length HCV clone in the pCR®-XL-TOPO® vector from Example 1 is digested with a unique restriction enzyme and cloned downstream of the pol promoter in the baculovirus vector, pFastBacDual® (Invitrogen, Inc.®, Carlsbad, Calif.), using In-Fusion® PCR (Stratagene®, San Diego, Calif.). A vector map of a full-length HCV clone in a pFastbac-Dual vector is shown in FIG. 4. The plasmid DNA is isolated from recombinant clones, and validated by sequencing the cloning junctions before generating baculovirus stock.

### Example 3

### Cloning the Structural Gene(s) of HCV in the Recombinant Baculovirus from Example 2 Under the Control of the p10 Promoter

In order to enhance the production of HCV virions in the insect cells, a sub-genomic component comprising the structural genes of HCV representing the core protein, E1 and E2 proteins, is cloned into the pFastbac-Dual vector under the control of the p10 promoter. The cloning is done in several steps. First, the structural genes of HCV are amplified from the plasmid DNA of the HCV full-length clone from Example 1 by PCR. The PCR amplified DNA is then cloned into pGL3-basic® vector (Promega® Corp., Madison, Wis.). The recombinant clones are verified by sequencing before digesting with a restriction enzyme targeting a unique restriction site in the vector. The HCV structural genes are then cloned downstream of P10 promoter in the pFastBac-Dual® vector by In-Fusion® PCR (Stratagene®, San Diego, Calif.). Recombinant clones are verified by sequencing before generating the recombinant baculovirus.

### Example 4

### Cloning the HCV Protease Gene in the Recombinant Baculovirus from Example 3 Under the Control of a Shrimp Virus Promoter p2

In order to further improve the production of HCV virions in the insect cells, HCV protease gene is sub-cloned downstream of the HCV structural genes in the plasmid vector from Example 3 under the control of a shrimp virus promoter p2 (Dhar et al., 2010 (Dhar, A. K., Kaizer, K. N., Lakshman, D. K. 2010. Transcriptional analysis of Penaeus stylirostris densovirus genes, Virology, In Press). The cloning is done in several steps. First, the HCV protease gene is amplified from the plasmid DNA of HCV full-length clone from Example 1 by PCR and the PCR amplified DNA is cloned into pGL3-basic vector (Promega Corp.®, Madison, Wis.) downstream of a p2 promoter (Dhar et al., 2007). The recombinant clones are verified by sequencing before digesting with a restriction enzyme targeting a unique restriction site in the vector. The p2 promoter driven HCV protease gene is then sub-cloned downstream of HCV structural genes in the pFastBac-Dual® vector by In-Fusion® PCR (Stratagene®, San Diego, Calif.). Recombinant clones are verified by sequencing before generating baculovirus.

### Example 5

### Transfection of Insect Cells (Sf9) Using Baculovirus Constructs from the Examples 2, 3, and 4

Recombinant baculovirus carrying either (i) a full-length HCV genome under the control of the pol-promoter (from Example 2), or (ii) a full-length HCV genome (pot-promoter) and the sub-genomic structural component of HCV under control of the p10-promoter (from Example 3), or (iii) a full-length HCV genome (under pol-promoter), the structural genes (under p10-promoter) and the protease gene (under p2 promoter) (from Example 4) are generated using a Bac-to-Bac® system (Invitrogen, Inc.®). Recombinant colonies from are picked and verified by restreaking on selective medium. Subsequently, recombinant colonies are taken to seed broth cultures and prepare midi-preparations to isolate baculovirus Bacmid® DNA. Sf9 cells are then transfected with the baculovirus Bacmid DNA using CellFectin® reagent (Invitrogen®, Carlsbad, Calif.). At 72 hours post-transfection, cell supernatants containing baculovirus particles are collected for subsequent amplification. In addition, at 72- and 96-hours post-transfection, cell supernatant as well as intact cells are collected for Western blot analysis to evaluate the expression of HCV RNA and proteins.

### Example 6

### Validation of the Expression of HCV Genomic RNA and HCV-Encoded Proteins in Sf9 Cells

Northern and Western blot analyses are performed to validate the expression of HCV genomic RNA and HCV-encoded proteins in Sf9 cells from the Example 4. In order to determine the expression of HCV genomic RNA in Sf9 cells, total genomic RNA is isolated from 72 and 96-hour post-transfected Sf9 cells using TRI reagent (Molecular Research Laboratory®, Cincinnati, Ohio), digested with DNase I, and electrophoresed in a 1% formaldehyde agarose gel. The RNA is transferred from the agarose gel to a nitrocellulose membrane, and hybridized to a biotin-labeled HCV probe. To determine the expression level of HCV encoded proteins, a total protein fraction is isolated from the 72- and 96-hours post-transfected Sf9 cells. Total proteins are separated by electrophoresis in a 12% polyacrylamide gel, transferred onto a nylon membrane, and probed with anti-HCV antibodies.

### Example 7

### Amplification of Baculovirus Carrying a Full-Length HCV Genome and Baculovirus Carrying a Full-Length HCV Genome and Sub-Genomic Fragments

Cell supernatants from Sf9 cells from Example 5 are amplified in a 50 ml culture flask. Sf9 cell cultures at a concentration of 2×10⁶ cells/ml are infected at ˜0.05 MOI with the transfected cell supernatants from Example 5. At 72 hours post-infection, cell supernatants are collected by centrifugation and baculovirus titers determined by plaque assay. The amplified Sf9 cell supernatant fractions are used to infect monolayers of Sf9 cells at dilutions of 10⁻⁶ to 10⁻⁹ of the original concentration. Infected cells are overlaid with agar and evaluated for plaque formation at 5 days post-infection. The baculovirus titer should range from 5×10⁷ to 1×10⁸ pfu/ml. This stock of baculovirus is used to infect Sf9 cells for the production of recombinant HCV and recombinant baculovirus.

### Example 8

### Production and Purification of HCV and Recombinant Baculovirus from the Sf9 Transfected Cells of Example 5

Monolayers of Sf9 cells are infected with either recombinant baculovirus containing a full-length HCV genome under control of the pol-promoter, or recombinant baculovirus carrying a full-length HCV genome and sub-genomic fragments (as produced in Examples 3 and 4). Cells are infected at an MOI of 10 and incubated for 72 hours to generate viral particles. Cell supernatants are removed and the remaining cells are lysed by freeze-thaw in phosphate buffer saline. Virus particles are then isolated from supernatants as well as from cell lysates using a sucrose density gradient centrifugation. Two viral bands are obtained; one band containing predominantly recombinant baculovirus, and the other band containing predominantly HCV.

The total yield of HCV from Sf9 cells infected with a baculovirus containing a full-length HCV genome is typically less than the HCV yield from those infected with a baculovirus carrying a full-length HCV genome plus the sub-genomic fragments because the HCV genome is translated as a single polypeptide that undergoes proteolytic cleavage to make mature virion protein. Sf9 cells being a non-host for HCV, the proteolytic processing of HCV polypeptide may be less efficient in Sf9 cells. As a result, the abundance of mature capsid protein in Sf9 cells infected with a baculovirus containing a full-length HCV genome may be less than the abundance of capsid protein in Sf9 cells that are infected with a baculovirus that not only contains a full-length HCV genome but also contains HCV capsid gene under the control of a different promoter.

### Example 9

### Characterization of HCV Isolated from the Transfected Cells of Example 7 by Transmission Electron Microscopy, SDS PAGE, and Glycosylation Pattern

Transmission electron microscopy (TEM) is performed to determine the morphology and size of the recombinant HCV isolated from Sf9 cells and generated via baculovirus-mediated infection. The HCV virions produced by the instant invention are structurally similar to native HCV. In addition, Sf9 cells infected with recombinant baculovirus are sampled from Example 7 and subjected to TEM analysis to determine the location of HCV assembly (nucleus or cytoplasm) in Sf9 cells. An aliquot of purified HCV virions produced by the instant invention as described in the Example 7 is boiled in a denaturing buffer containing 0.5% sodium dodecyl sulfate (SDS) and 1%13-mercaptoethanol for 10 minutes, and subjected to SDS polyacrylamide gel electrophoresis (SDS-PAGE) on 12% gels. The proteins from the gel are transferred to nylon membrane and the membrane is probed with anti-HCV antibody to demonstrate the reactivity of the purified HCV to homologous antibody.

Purified virions are pre-treated with 10 U endoglycosidase (Endo) H (New England Biolabs, Beverly, Mass.) per microgram of protein at 37° C. for 3 hours in a buffer containing 50 mmol/L sodium citrate at pH 5.5 prior to denaturation and separation using SDS-PAGE to remove any glycosylation from the viral coat proteins. Untreated purified wild-type HCV sample serves as a control. The comparison of HCV peptide profiles upon treatment of endoglycosidase vs. control sample is used to determine the extent of glycosylation of the Sf9 generated HCV relative to that of the native HCV. Comparison of deglycosylated control HCV proteins with deglycosylated HCV proteins produced by the instant invention using SDS-PAGE establishes that the HCV proteins produced using the instant invention are processed accurately (i.e., to the same molecular weight as in the native HCV).

Alternatively, purified virions are analyzed by MALDI TOF mass spectrometry. The molecular masses of virion proteins are determined by matrix assisted laser desorption ionization time-of-flight mass spectrometry (MALDITOF MS). Samples are prepared for MALDI-MS analysis as follows: 0.5 ml of matrix solution 1 (0.13 M 2,5-dihydroxybenzoic acid [DHB] and 6 mM 5-methoxysalicyclic acid [MSA] in 50% acetone and 50% isopropanol) is applied to the virus sample and allowed to air dry until visible crystals are formed. The dried sample is washed with 10 ml of 0.1% trifluoroacetic acid (TFA). One microliter of matrix solution 2 (5 mM DHB, 2.5 mM MSA in 30% acetonitrile, 0.1% TFA) is subsequently applied to the sample and allowed to air dry. Positive-ion mass spectra are subsequently acquired with a delayed extraction system in reflectron mode. The following instrument parameters are used: 25-kV source potential, 23.8-kV extraction grid potential, and 275-V guide wire potential. Calibration of the mass scale is accomplished with bovine serum albumin (Boehringer Mannheim, Indianapolis®, Ind.). The average mass/charge (m/z) values of the peaks are determined with an appropriate software package such as the Voyager® Elite software package (PerSeptive Biosystems®).

### Example 10

### Establishing the Antigenicity of HCV Isolated from Example 7

The antigenic authenticity of the HCV produced by the instant invention is tested by its ability to produce antibodies in mice that cross react with native HCV, and its seroreactivity to plasma from patients infected with HCV.

### Example 11

### Establishing the Infectivity of HCV Isolated from Example 7 by In Vitro Assay

The ability of the HCV produced by the instant invention to infect mammalian cells is demonstrated by the ability of the purified virions produced in Example 7 to replicate and propagate in a human hepatoma cell line (Huh-7). Cytopathic effects upon infection by purified HCV are recoded and, the expression of immune genes known to be modulated by HCV infection (e.g., MxA, IKBKbeta, CREBBP) are measured by real-time RT-PCR and compared to the expression profile of homologous genes in uninfected cells.

### Example 12

### Production of an HCV Vaccine by Inactivation of the Purified Infectious Virus

The purified infectious virus produced in Example 8 is inactivated by treatment with formalin at a ratio of 1:5000 (formalin:virus concentrate) for 18 hr at 37° C. The mixture is then filtered through a 1 micron filter and dialyzed for 4 hr at 4° C. with 0.01M phosphate buffered saline at pH 7.2 to remove residual formalin. The final composition is used directly as a vaccine or combined with an adjuvant to improve efficacy.

### Example 13

### Production of Infectious HCV in Mammalian Cell Culture Using a Single Baculovirus and its Conversion to a Vaccine

The purified recombinant baculovirus produced according to Example 8 is added to cultured Chinese Hamster Ovary (CHO) cells at an MOI of 10 and incubated 72 hours to generate viral particles. Cell supernatants are removed and cells are lysed by freeze-thaw in 0.01M phosphate buffered saline (PBS) at pH 7.2. Virus particles are isolated from supernatants as well as from cell lysates using a sucrose density gradient centrifugation. One band is observed in the density gradient that contains virions with a similar in morphology to the wild type HCV. This band represents infectious HCV and is inactivated with formalin according to Example 12. Following filtration through a 0.1 micron filter) and dialysis against PBS, the resulting composition is used directly, or in combination with an adjuvant, as a vaccine.

### Example 14

### Production of Infectious TSV in Insect Cell Culture Using Baculovirus and Conversion to a Vaccine

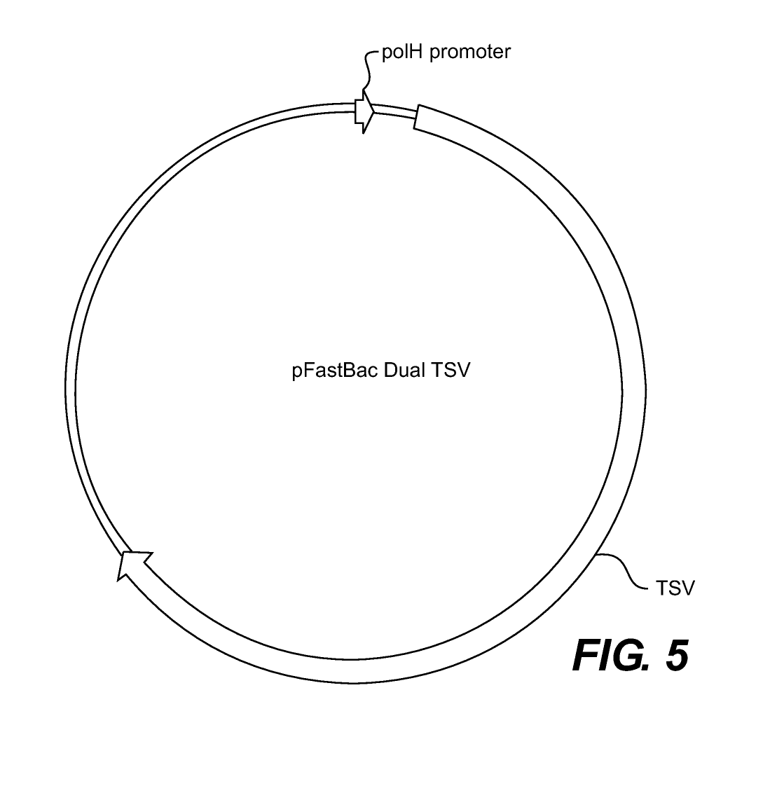

A PCR-amplified cDNA of a full-length genome of Taura Syndrome Virus (TSV) is prepared and cloned into a pCR®-XL-TOPO® vector as described in Example 1. The plasmid DNA of the full-length TSV clone in the pCR®-XL-TOPO® vector is digested with a unique restriction enzyme and cloned downstream of the pol promoter in the baculovirus vector, pFastBacDual® (Invitrogen, Inc.®, Carlsbad, Calif.), using In-Fusion® PCR (Stratagene, San Diego, Calif.) (FIG. 5).

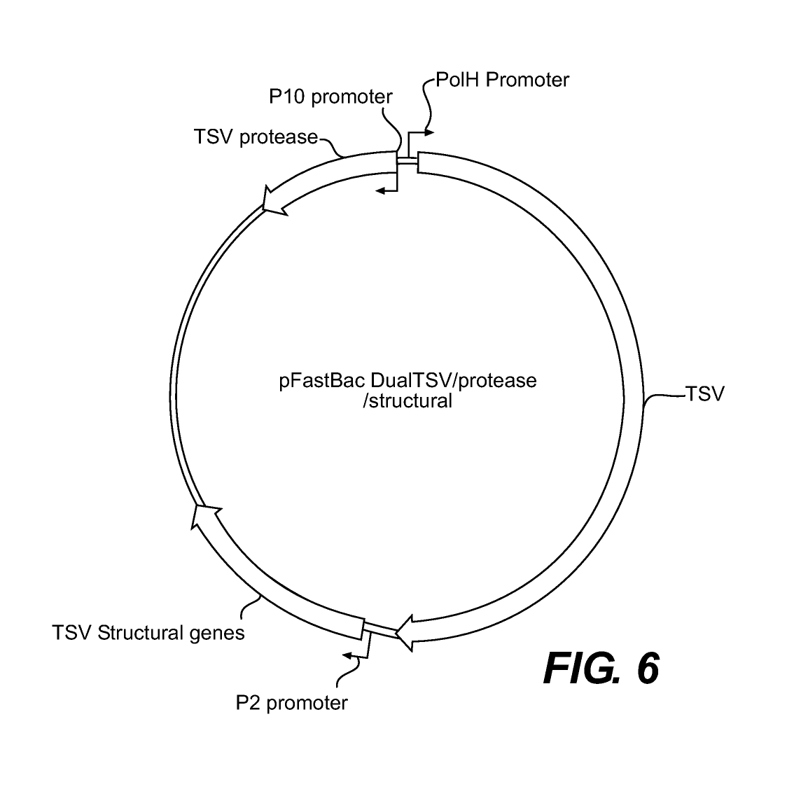

In order to enhance the production of TSV virions in the insect cells, sub-genomic components comprising the protease domain and ORF-2 of the TSV are cloned into the pFastBacDual® vector under the control of the p10 promoter and the p2 baculovirus promoter, respectively. This is accomplished in three steps. First, p2 promoter is inserted by In-Fusion® PCR (Clonetech, Inc.®) into the pFastBacDual® vector (Invitrogen®) already containing the full-length TSV genome using XhoI and resulting in the placement of the p10 promoter downstream of the p2 baculovirus promoter in the pFastBacDual® vector. Next, TSV protease domain is amplified by PCR from a full-length TSV clone and inserted by In-Fusion PCR® (Clonetech, Inc.®) into to the pFastBacDual® vector construct under control of the p10 promoter. Finally, TSV ORF2 is amplified by PCR from a full-length TSV clone and inserted by In-Fusion PCR® (Stratagene®) downstream of the p2 promoter. A schematic diagram of a full-length TSV clone and the sub-genomic components of TSV in a pFastBacDual® vector is shown in FIG. 6.

Recombinant baculovirus carrying the full-length TSV genome under control of the pol-promoter and the TSV protease domain under control of the p10-promoter and ORF-2 under control of the p2-promoter is generated using a Bac-to-Bac® system (Invitrogen, Inc.®) using the pFastBacDual® vector described in FIG. 5. Recombinant colonies, picked and verified by re-streaking on selective medium, are taken to seed broth cultures to prepare midi-preparations to isolate baculovirus Bacmid® DNA. Sf9 insect cells are then transfected with the baculovirus Bacmid® DNA using CellFectin® reagent (Invitrogen®, Carlsbad, Calif.). Cells are infected at an MOI of 10 and incubated for 72 hours to generate viral particles. Cell supernatants are removed and the remaining cells are lysed by freeze-thaw in phosphate buffer saline. Virus particles are then isolated from supernatants as well as from cell lysates using a sucrose density gradient centrifugation. Two viral bands are obtained; one band containing predominantly recombinant baculovirus, and the other band containing predominantly TSV. The purified TSV is then inactivated by incubation with formalin as in Example 12, and used as such as a vaccine.

### Example 15

### Production of Infectious PRRSV in Insect Cell Culture Using Baculovirus and Conversion to a Vaccine

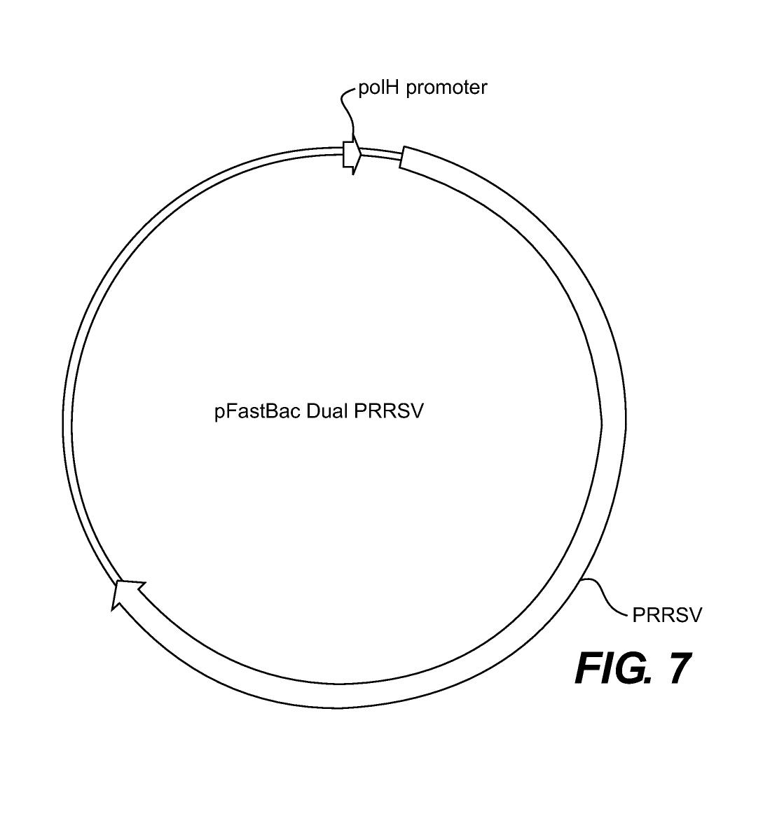

A PCR-amplified cDNA of a full-length genome of Porcine Reproductive and Respiratory Syndrome Virus (PRRSV) can be amplified by RT-PCR from infected porcine tissue and cloned into a pCR®-XL-TOPO® vector as described in Example 1. The plasmid DNA of the full-length PRRSV clone in the pCR®-XL-TOPO® vector is digested with a unique restriction enzyme and cloned downstream of the pol promoter in the baculovirus vector, pFastBacDual® (Invitrogen, Inc.®, Carlsbad, Calif.), using In-Fusion® PCR (Stratagene®, San Diego, Calif.). In order to enhance the production of PRRSV virions in the insect cells, a sub-genomic component comprising the ORF-2 (or 1) through ORF-7 (approximately 9 kb) of the PRRSV genome is cloned into the pFastBacDual® vector under the control of the p10 promoter. A vector map of a full-length PRRSV genome and the sub-genomic component of PRRSV in a pFastBacDual® vector is shown in FIG. 7.

Recombinant baculovirus carrying the full-length PRRSV genome under control of the pol-promoter and the sub-genomic structural components of PRRSV ORF-1 through ORF-7 under control of the p10-promoter is generated using a Bac-to-Bac® system (Invitrogen, Inc.)® using the pFastBacDual vector described in FIG. 6. Recombinant colonies, picked and verified by re-streaking on selective medium, are taken to seed broth cultures to prepare midi-preparations to isolate baculovirus Bacmid DNA. Sf9 insect cells are then transfected with the baculovirus Bacmid DNA using CellFectin reagent (Invitrogen, Carlsbad, Calif.). Cells are infected at an MOI of 10 and incubated for 72 hours to generate viral particles. Cell supernatants are removed and the remaining cells are lysed by freeze-thaw in phosphate buffer saline. Virus particles are then isolated from supernatants as well as from cell lysates using a sucrose density gradient centrifugation. Two viral bands are obtained; one band containing predominantly recombinant baculovirus, and the other band containing predominantly PRRSV. The purified PRRSV is then inactivated by incubation with formalin as in Example 12, and used as such as a vaccine.

### Example 16

### Production of Infectious WNV in Insect Cell Culture Using Baculovirus and Conversion to a Vaccine

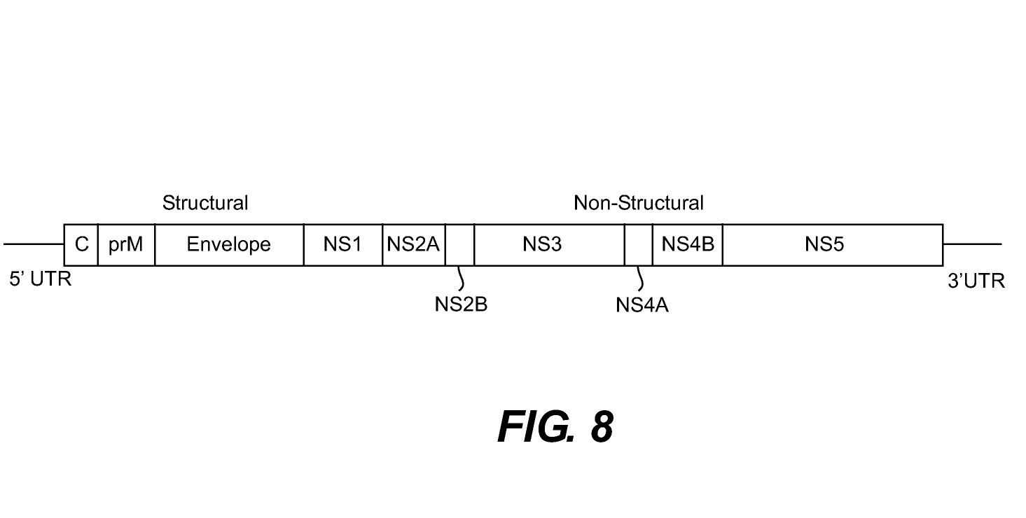

West Nile Virus (WNV) has widespread prevalence throughout the United States and other parts of North America. The expression of symptoms to an infection in humans can range from a mild fever and headache to a life threatening neuroinvasive disease in the form of encephalitis or meningitis. The genetic material of WNV is a positive-sense, single-strand of RNA, between 11,000 and 12,000 nucleotides, and encoding seven non-structural proteins and three structural proteins (FIG. 8)

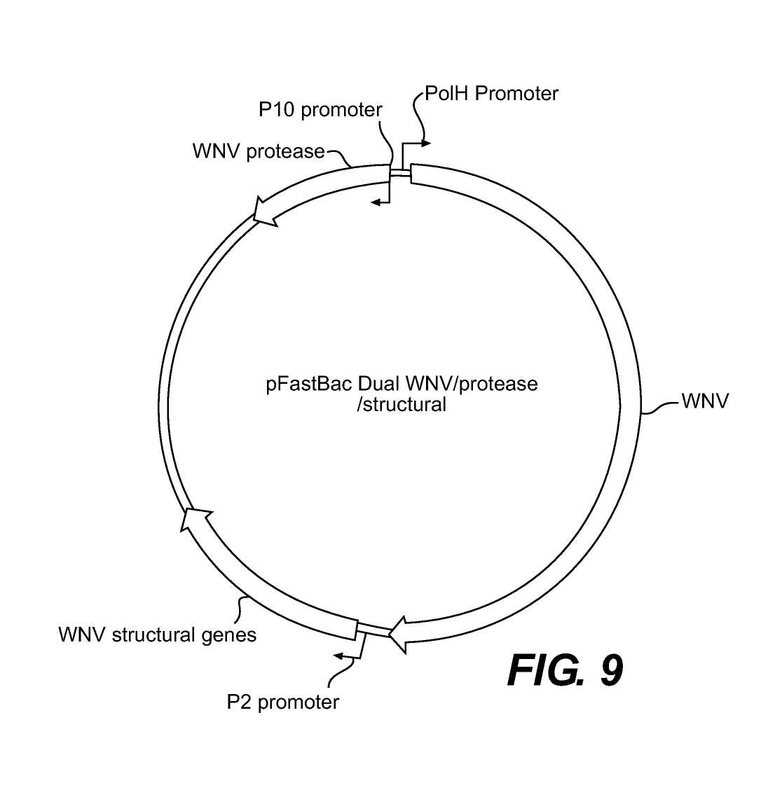

Currently there is no effective approved therapy or vaccine available for humans. To overcome this problem, a PCR-amplified cDNA of a full-length genome of WNV is prepared using the sequence from GenBank #M12294, and cloning it into a pCR®-XL-TOPO® vector as described in Example 1. Alternatively, a new infectious clone from any recent outbreak is prepared according to the method of Shi et al (2002), or Yamshchikov (U.S. Pat. No. 7,459,163) but using the pCR®-XL-TOPO® vector. U.S. Pat. No. 7,459,163 and Pei-Yong Shi, Mark Tilgner, Michael K. Lo, Kim A. Kent, and Kristen A. Bernard (2002) Infectious cDNA Clone of the Epidemic West Nile Virus from New York City. Journal of Virology, Vol 76, p. 5847-5856, hereby incorporated by reference in their entirety. The plasmid DNA of the full-length WNV clone in the pCR®-XL-TOPO® vector is digested with a unique restriction enzyme and cloned downstream of the pol promoter in the baculovirus vector, pFastBacDual® (Invitrogen, Inc.®, Carlsbad, Calif.), using In-Fusion® PCR (Stratagene®, San Diego, Calif.). In order to enhance the production of WNV virions in the insect cells, sub-genomic components comprising the first three genes (i.e., the structural genes) of the 5′ end of the WNV genome is cloned into the pFastBacDual® vector under the control of the p10 promoter. A schematic diagram of a full-length WSV genome and the sub-genomic component of WSSV in a pFastBacDual vector is shown in FIG. 9.

Recombinant baculovirus carrying the full-length WNV genome under control of the pot-promoter and the sub-genomic structural components of WNV structural protein genes under control of the p10-promoter is generated using a Bac-to-Bac® system (Invitrogen, Inc.®) using the pFastBacDual® vector described in FIG. 8. Recombinant colonies, picked and verified by re-streaking on selective medium, are taken to seed broth cultures to prepare midi-preparations to isolate baculovirus Bacmid® DNA. Sf9 insect cells are then transfected with the baculovirus Bacmid® DNA using CellFectin® reagent (Invitrogen®, Carlsbad, Calif.). Cells are infected at an MOI of 10 and incubated for 72 hours to generate viral particles. Cell supernatants are removed and the remaining cells are lysed by freeze-thaw in phosphate buffer saline. Virus particles are then isolated from supernatants as well as from cell lysates using a sucrose density gradient centrifugation. Two viral bands are obtained; one band containing predominantly recombinant baculovirus, and the other band containing predominantly WNV. The purified WNV is then inactivated by incubation with formalin as in Example 12, and used as such as a vaccine.

### Example 17

### Cloning of a Full-Length HCV Genome Under the Control of a Pol-Promoter Using the Baculovirus Expression Vector System (REVS)

The plasmid DNA of the full-length HCV clone in the pCR®-XL-TOPO® vector from Example 1 is digested with a unique restriction enzyme and cloned downstream of the pol promoter into a baculovirus transfer vector pVL1392/1393 (BD Bioscience, San Jose, Calif.). Subsequently, Sf9 cells are co-transfected with the recombinant transfer vector pVL1392/1393-HCV and a linearized AcNPV Baculovirus DNA (BD BaculoGold™) to generate recombinant baculovirus containing HCV genome (FIG. 9). Recombinant baculovirus is amplified to produce high titer stock, and used to infect Sf9 cells to purify HCV. The purified HCV is characterized as described in the Example 9. The antigenicity and infectivity of the purified HCV is established following Examples 10 and 11.

### Example 18

### Cloning of a Full-Length HCV Genome, HCV Structural Genes, and HCV Protease Gene Using the Baculovirus Expression Vector System (REVS)

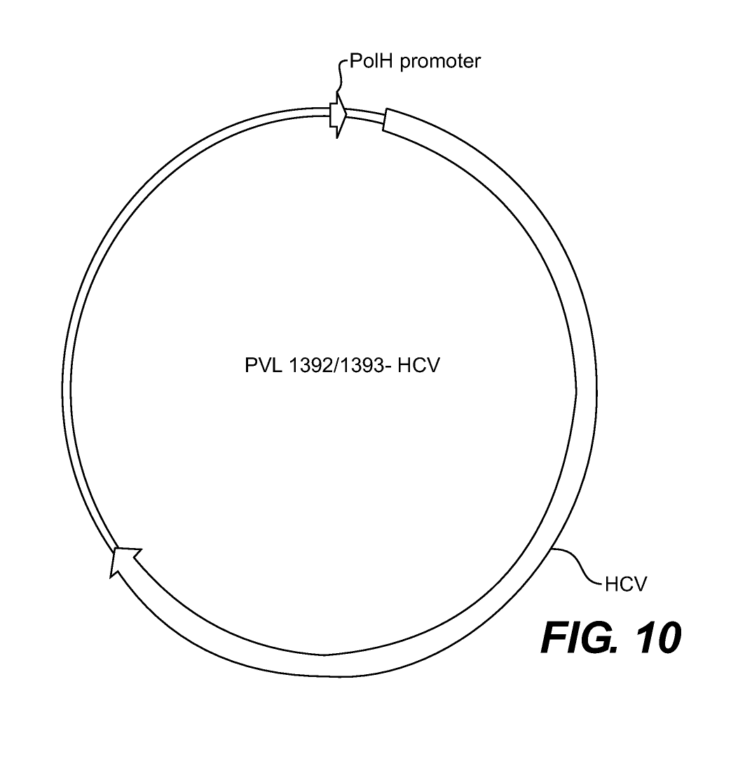

The full-length genome of HCV, the HCV structural genes under the control of p10 promoter, and the HCV protease gene under the control of p2 promoter is amplified by PCR from the plasmid DNA of the clone described in Example 4. The amplicon is cloned downstream of the pol promoter into a baculovirus transfer vector pVL1392/1393 (BD Bioscience, San Jose, Calif.) (FIG. 10). Subsequently, Sf9 cells are co-transfected with the recombinant transfer vector pVL1392/1393-HCV-Struc-Pro and a linearized AcNPV Baculovirus® DNA (BD BaculoGold™) to generate recombinant baculovirus containing HCV genome. Recombinant baculovirus is amplified to produce high titer stock, and to infect Sf9 cells to purify HCV. The purified HCV is characterized as described in the Example 9. The antigenicity and infectivity of the purified HCV is established following Examples 10 and 11.

### Example 19

### Cloning of a Full-Length HCV Genome Under the Control of a Pol-Promoter Using the Sapphire™ Baculovirus DNA

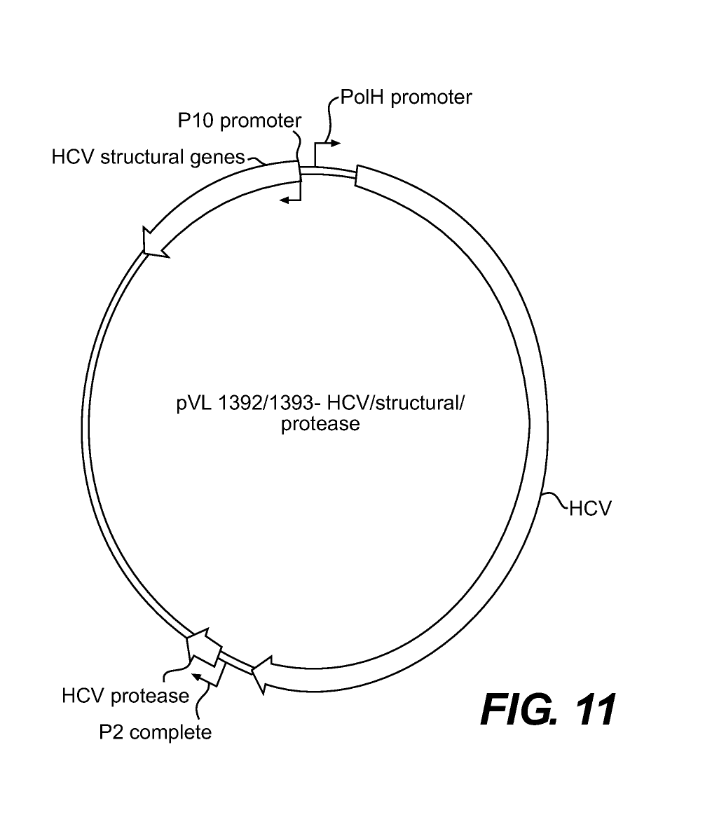

The plasmid DNA of the full-length HCV clone in the pCR®-XL-TOPO® vector from Example 1 is digested with a unique restriction enzyme and cloned downstream of the pol promoter into a baculovirus transfer vector pORB (Orbigen, San Diego, Calif.) under the control of pol promoter (FIG. 11). Subsequently, Sf9 cells are co-transfected with the recombinant transfer vector pORB-HCV and Sapphire™ Baculovirus DNA (Orbigen) using Sapphire Insect transfection kit (Orbigen) to generate recombinant baculovirus containing HCV genome. Recombinant baculovirus is amplified to produce high titer stock, and to infect Sf9 cells to purify HCV. The purified HCV is characterized as described in the Example 9. The antigenicity and infectivity of the purified HCV is established following Examples 10 and 11.

### Example 20

### Cloning of a Full-Length HCV Genome, HCV Structural Genes, and HCV Protease Gene Using the Sapphire™ Baculovirus DNA

The full-length genome of HCV, the HCV structural genes under the control of p10 promoter and the HCV protease gene under the control of p2 promoter is amplified by PCR from the plasmid DNA of the clone described in Example 4. The amplicon can be cloned downstream of the pol promoter into a baculovirus transfer vector pORB (Orbigen®, San Diego, Calif.) under the control of pol promoter (FIG. 11). Subsequently, Sf9 cells can be co-transfected with the recombinant transfer vector pORB-HCV and Sapphire™ Baculovirus DNA (Orbigen®®) using Sapphire™ Insect transfection kit (Orbigen®) to generate recombinant baculovirus containing HCV genome. Recombinant baculovirus is amplified to produce high titer stock, and to infect Sf9 cells to purify HCV. The purified HCV can be characterized as described in the Example 9. The antigenicity and infectivity of the purified HCV can be established following Examples 10 and 11.

### Example 21

### Cloning of a Full-Length HCV Genome in pBac Vector

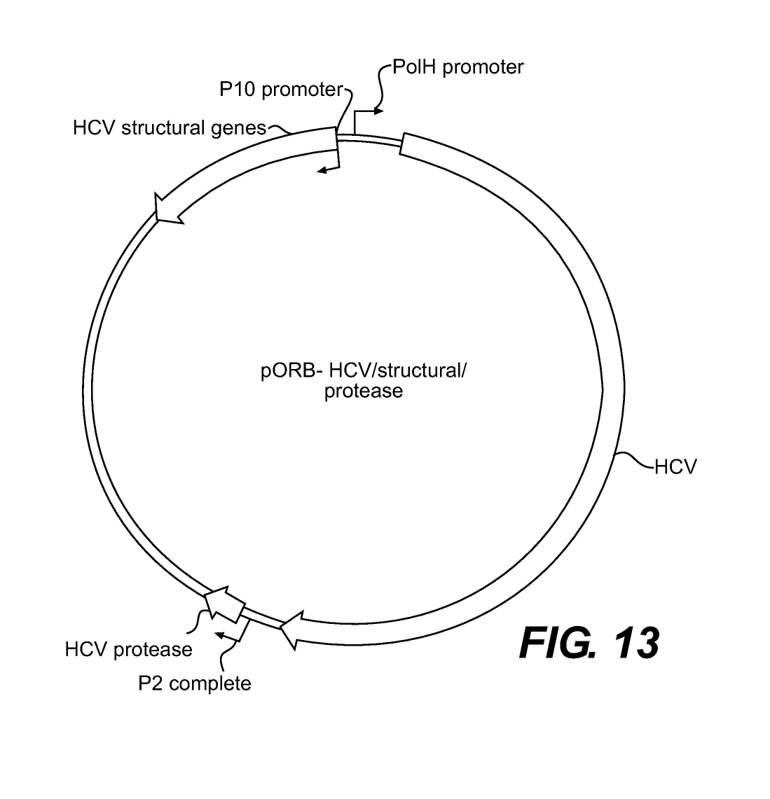

The plasmid DNA of the full-length HCV clone in the pCR®-XL-TOPO® vector from Example 1 is digested with a unique restriction enzyme and cloned downstream of the pol promoter into a pBAC™ 1-3 vector or cloned downstream of gp64 promoter into pBAC5 or pBAC-6 baculovirus transfer vector (Novagen, Madison, Wis.) (FIG. 13). Subsequently, Sf9 cells can be co-transfected with the recombinant transfer vector pBAC™ 1-3 or pBAC5 or pBAC-6 using BacVector® Transfection kit (Novagen) to generate recombinant baculovirus containing HCV genome. Recombinant baculovirus is amplified to produce high titer stock, and to infect Sf9 cells to purify HCV. The purified HCV can be characterized as described in the Example 9. The antigenicity and infectivity of the purified HCV can be established following Examples 10 and 11.

### Example 22

### Cloning of a Full-Length HCV Genome, HCV Structural Genes, and HCV Protease Gene Using a pBAC Vector

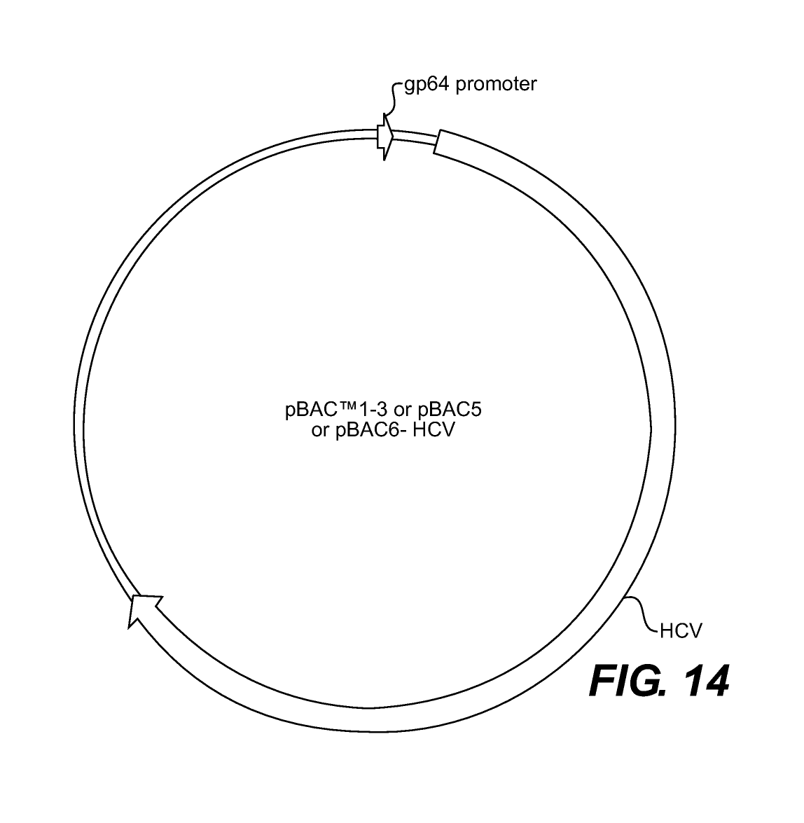

The full-length genome of HCV, the HCV structural genes under the control of p10 promoter and the HCV protease gene under the control of p2 promoter is amplified by PCR from the plasmid DNA of the clone as described in Example 4. The amplicon is cloned downstream of the pol promoter into a pBAC™ 1-3 vector or cloned downstream of gp64 promoter into pBAC5 or pBAC-6 baculovirus transfer vector (Novagen®, Madison, Wis.) (FIG. 14). Subsequently, Sf9 cells are co-transfected with the recombinant transfer vector pBAC™ 1-3 or pBAC5 or pBAC-6 BacVector® Transfection kit (Novagen®) to generate recombinant baculovirus containing HCV genome. Recombinant baculovirus is amplified to produce high titer stock, and to infect Sf9 cells to purify HCV. The purified HCV is characterized as described in the Example 9. The antigenicity and infectivity of the purified HCV is established following Examples 10 and 11.

### Example 23

### Cloning of a Full-Length HCV Genome Using a BestBac Expression Systems

The plasmid DNA of the full-length HCV clone in the pCR®-XL-TOPO® vector from Example 1 is digested with a unique restriction enzyme and cloned downstream of the pol promoter into a pVL1292/pVL1293 vector (Expression Systems LLC®). Subsequently, Sf9 cells are co-transfected with the recombinant transfer vector pVL1292/pVL1293-HCV and a BestBac® v-cath/chit deleted DNA—a linearized viral backbone (Expression Systems LLC®) to generate recombinant baculovirus containing HCV genome. Recombinant baculovirus is amplified to produce high titer stock, and to infect Sf9 cells to purify HCV. The purified HCV is characterized as described in the Example 9. The antigenicity and infectivity of the purified HCV is established following Examples 10 and 11.

### Example 24

### Cloning of a Full-Length HCV Genome, HCV Structural Genes, and HCV Protease Gene Using a BestBac Expression Systems

The full-length genome of HCV, the HCV structural genes under the control of p10 promoter and the HCV protease gene under the control of p2 promoter is amplified by PCR from the plasmid DNA of the clone described in Example 4. The amplicon is cloned downstream of the pol promoter into a into a pVL1292/pVL1293 vector (Expression Systems LLC). Subsequently, Sf9 cells are co-transfected with the recombinant transfer vector pVL1292/pVL1293-HCV and a BestBac v-cath/chit deleted DNA—a linearized viral backbone (Expression Systems LLC) to generate recombinant baculovirus. Recombinant baculovirus is amplified to produce high titer stock, and to infect Sf9 cells to purify HCV. The purified HCV is characterized as described in the Example 9. The antigenicity and infectivity of the purified HCV is established following Examples 10 and 11.

### Example 25

### Cloning of a Full-Length HCV Genome Under the Control of a Mammalian Promoter or a Pantropic Promoter

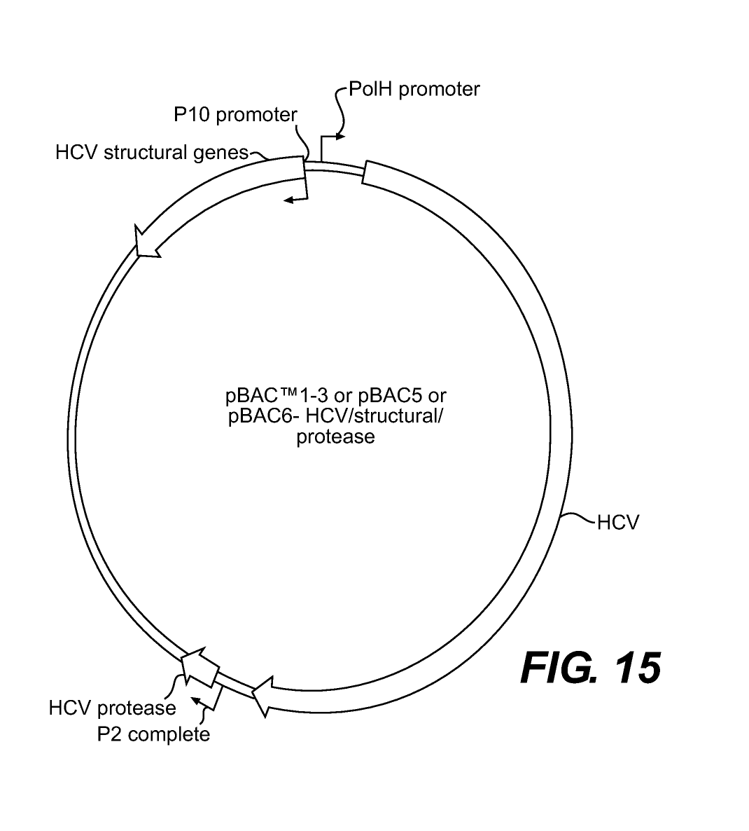

The polyhedrin promoter in a baculovirus transfer vector, such as pFastBacDual® (Invitrogen®) is replaced with a mammalian promoter cytomegalovirus (CMV) promoter, a thymidine kinase (TK) promoter, or a pantropic promoter such as the P2 promoter of infectious hypodermal hematopoietic necrosis virus (IHHNV) (PA41 incorporated by reference), or the ie1 promoter of white spot syndrome virus of shrimp. The plasmid DNA of the full-length HCV clone in the pCR®-XL-TOPO® vector from Example 1 is digested with a unique restriction enzyme and cloned downstream of the CMV promoter/CMV ie enhancer, or TK or P2 or ie1 promoters into a pFastBacDual by In-Fusion PCR (FIG. 15). Recombinant colonies from are picked and verified by restreaking on selective medium. Subsequently, recombinant colonies are taken to seed broth cultures and prepare midi-preparations to isolate baculovirus Bacmid DNA. Sf9 cells are then transfected with the baculovirus Bacmid DNA using CellFectin reagent (Invitrogen, Carlsbad, Calif.) to generate recombinant baculovirus. Recombinant baculovirus is then amplified to produce high titer stock, and to infect mammalian cells (e.g. CHO, 293T etc.) to purify HCV. The purified HCV is characterized as described in the Example 9. The antigenicity and infectivity of the purified HCV isolated from mammalian cells is established following Examples 10 and 11.

### Example 26

### Production and Purification of HCV and Recombinant Baculovirus by Transfection of a Humanized Insect Cell Line(s) Modified to Produce Humanized Protein Glycosylation Patterns

Monolayers of humanized insect cell line(s) obtained according to Kost, T. A., Condreay, J. P., and Jarvis, D. L. Nature Biotechnology 23:567-575; 2005 are infected with either recombinant baculovirus containing a full-length HCV genome under control of the pol-promoter, or recombinant baculovirus carrying a full-length HCV genome and sub-genomic fragments (as produced in Examples 3 and 4). Cells are infected at an MOI of 10 and incubated for 72 hours to generate viral particles as described in Example 8. Cell supernatants are removed and the remaining cells are lysed by freeze-thaw in phosphate buffer saline. Virus particles are then isolated from supernatants as well as from cell lysates using a sucrose density gradient centrifugation. Two viral bands are obtained; one band containing predominantly recombinant baculovirus, and the other band containing predominantly HCV. The mature HCV virions so produced will contain envelope proteins E1 and E2 that exhibit a humanized glycosylation pattern.

1. A baculovirus capable of infecting a facilitating host comprising a DNA sequence that codes for a functional positive sense single stranded RNA viral genome (“(+)sense RNA”) that is not capable of infecting said facilitating host, wherein the transcription of said DNA sequence is under the control of a single promoter.

2. The baculovirus of claim 1, wherein said genome of said functional (+)sense RNA virus will not replicate in said facilitating host unless part of the genome of said baculovirus.

3. The baculovirus of claim 1 wherein said facilitating host is an insect cell.

4. The baculovirus of claim 1 wherein said single promoter is a promoter functional in an insect cell.

5. The baculovirus of claim 1, wherein said host of said functional (+)sense RNA virus is a human cell.

6.-8. (canceled)

9. The baculovirus of claim 1, wherein said DNA sequence codes for a structural and non-structural genes sufficient for infection in its host cell.

10.-11. (canceled)

12. The baculovirus of claim 1 further comprising a second DNA sequence that codes for a sub-genomic component of said functional (+)sense RNA virus.

13. The baculovirus of claim 12, wherein said second DNA sequence further comprises a second promoter.

14.-22. (canceled)

23. A composition comprising a first virus capable of infecting a facilitating host comprising a first DNA sequence that codes for a (+)sense RNA viral genome or complement thereof that is not capable of infecting said facilitating host and a second DNA sequence coding for a baculovirus genome capable of infecting said facilitating host, wherein the expression of said first DNA sequence is under the control of a single promoter.

24. The composition of claim 23, wherein said second DNA sequence further comprises a third DNA sequence that codes for a nonstructural gene of said (+)sense RNA virus.

25. (canceled)

26. The composition of claim 23, wherein said (+)sense RNA virus is Hepatitis C Virus.

27. The composition of claim 23, wherein said (+)sense RNA virus is West Nile Virus.

28. The composition of claim 23, wherein said (+)sense RNA virus is PRRS Virus.

29. The composition of claim 23, wherein said (+)sense RNA virus is in the Flaviviridae family.

30. The composition of claim 23, wherein a host cell for said (+)sense RNA virus is a human cell.

31. The composition of claim 23, wherein the (+)sense RNA virus is in the Arbovirus family.

32. The composition according to claim 9, wherein a host cell for said (+)sense RNA virus is a mammalian cell.

33. A method of amplifying a (+)sense RNA viral genome comprising
a) infecting a facilitating host cell with virus comprising a DNA molecule comprising a first DNA sequence coding for a viral genome of said facilitating host cell and a second DNA sequence coding for a (+)sense RNA viral genome that does not infect said facilitating host cell, wherein said second DNA sequence is operably linked to only one promoter;
b) obtaining the supernatant, cell lysate, or supernatant and cell lysate of said infected facilitating host cell;
c) transducing a host cell of the (+)sense RNA virus with said supernatant, cell lysate, or supernatant and cell lysate.

34.-39. (canceled)

40. A vaccine comprising a (+)sense RNA viral genome produced by the method of
a) infecting a facilitating host cell with a virus comprising a DNA molecule comprising a first DNA sequence coding for a viral genome of said facilitating host cell and a second DNA sequence coding for a (+)sense RNA viral genome that does not infect said facilitating host, wherein said second DNA sequence is operably linked to only one promoter;
b) obtaining the supernatant, cell lysate, or supernatant and cell lysate of said facilitating host cell; and
c) transducing a host cell of the (+)sense RNA with said supernatant, cell lysate, or supernatant and cell lysate, wherein the (+)sense RNA virus is inactivated or attenuated before use.

41. (canceled)

42. A vaccine comprising a (+)sense RNA virus that is not capable of infecting a facilitating host comprising a viral envelope comprising proteins derived from said facilitating host.

43.-45. (canceled)
